(19) United States
(12) Patent Application Publication
(10) Pub. No.: US 2023/0293884 A1
(43) Pub. Date: Sep. 21, 2023

(54) PASTE FOR PREPARING BIODEGRADABLE ELECTROCEUTICAL, BIODEGRADABLE ELECTRONIC DEVICE FORMED USING SAME AND MANUFACTURING METHOD THEREFOR, AND BIODEGRADABLE ELECTROCEUTICAL AND PREPARATION METHOD THEREFOR

(71) Applicant: SEOUL NATIONAL UNIVERSITY R&DB FOUNDATION, Seoul (KR)

(72) Inventors: Seung Kyun KANG, Seoul (KR); Ju Yong LEE, Seoul (KR)

(21) Appl. No.: 18/015,956

(22) PCT Filed: Dec. 11, 2020
(86) PCT No.: PCT/KR2020/018179

§ 371 Date: Jan. 12, 2023

(30) Foreign Application Priority Data
Jul. 27, 2020 (KR) 10-2020-0093157
Oct. 30, 2020 (KR) 10-2020-0143559

(51) Int. Cl. A61N 1/05; A61L 27/04; A61L 27/58; A61L 27/50; B33Y 70/10; B33Y 80/00

(52) U.S. Cl. CPC A61N 1/0556; A61L 27/047; A61L 27/50; A61L 27/58; B33Y 70/10; B33Y 80/00; A61L 2300/802; B33Y 10/00

(57) ABSTRACT

Provided are a paste for simply and easily producing biodegradable electroceuticals capable of integrating electronic circuits to implement light, thin, short, and small characteristics and of being decomposed in the human body after a certain period of time so as not to require additional surgery, a biodegradable electronic device formed using the paste, and a method of producing the same. According to an embodiment of the present invention, the paste includes a functional inorganic powder providing conductive, semiconductive, dielectric, or insulating properties, a humectant, a matrix polymer, and an organic solvent.

### TECHNICAL FIELD

The present invention relates to an electroceutical, and more particularly, a paste for producing biodegradable electroceuticals capable of being self-decomposed in the human body, a biodegradable electronic device formed using the paste, a biodegradable electroceutical, and a method of producing the same.

### BACKGROUND ART

Currently, life expectancy is increasing and medical technology is being remarkably developed. In particular, research on new electroceuticals is increasing. Electroceutical is a compound word of electronic and pharmaceutical, and it may provide a therapeutic effect by controlling nerve functions by using electrical energy. Existing drugs flow along blood vessels and thus may cause side effects in sites where treatment is not desired, but electroceuticals select specific sites requiring treatment and thus are relatively safe.

Existing electroceuticals or nerve stimulators have the following limitations. First, a semipermanent material is used for production and thus additional surgery for removing it from the human body is required after treatment. Second, due to a complicated wire configuration for an electronic circuit, great inconvenience is caused when inserted into the human body. Third, an electronic device corresponding to a complicated shape of the human body may be accompanied by difficulty in production and degradation in function. Fourth, electrodes and a nerve stimulator implanted in the human body require a large invasion space.

### DETAILED DESCRIPTION OF THE INVENTION

### Technical Problem

The present invention provides a paste for simply and easily producing biodegradable electroceuticals capable of integrating electronic circuits to implement light, thin, short, and small characteristics and of being decomposed in the human body after a certain period of time so as not to require additional surgery, and a method of producing the same.

The present invention also provides a biodegradable electronic device formed using the paste, and a method of producing the same.

The present invention also provides a biodegradable electroceutical capable of integrating electronic circuits to implement light, thin, short, and small characteristics and of being decomposed in the human body after a certain period of time so as not to require additional surgery, and a method of producing the same.

However, the scope of the present invention is not limited thereto.

### Technical Solution

According to an aspect of the present invention, there is provided a paste for simply and easily producing biodegradable electroceuticals, a biodegradable electronic device, and a method of producing the same.

According to an embodiment of the present invention, the paste for producing biodegradable electroceuticals may include a functional inorganic powder providing conductive, semiconductive, dielectric, or insulating properties, a humectant, a matrix polymer, and an organic solvent.

According to an embodiment of the present invention, the functional inorganic powder may provide a conductive function, and include one or more selected from the group consisting of magnesium (Mg), iron (Fe), zinc (Zn), molybdenum (Mo), tungsten (W), calcium (Ca), potassium (K), sodium (Na), silicon (Si), amorphous indium gallium zinc oxide (a-IGZO), germanium (Ge), and alloys thereof.

According to an embodiment of the present invention, the functional inorganic powder may provide a conductive function, and a volume fraction of the functional inorganic powder with respect to a total volume of the paste may range from 0.35 to 0.41.

According to an embodiment of the present invention, the functional inorganic powder may provide a conductive function, and the paste may include 1 g to 15 g of the functional inorganic powder, 0.1 ml to 1 ml of the humectant, and 0.05 g to 1 g of the matrix polymer, per 1 ml of the organic solvent.

According to an embodiment of the present invention, the functional inorganic powder may provide a semiconductive function, and include one or more selected from the group consisting of Si, Ge, zinc oxide (ZnO), and aluminum-doped zinc oxide (AZO).

According to an embodiment of the present invention, the functional inorganic powder may provide a semiconductive function, and a volume fraction of the functional inorganic powder with respect to a total volume of the paste may range from 0.17 to 0.23.

According to an embodiment of the present invention, the functional inorganic powder may provide a semiconductive function, and the paste may include 1 g to 5 g of the functional inorganic powder, 0.1 ml to 1 ml of the humectant, and 0.05 g to 1 g of the matrix polymer, per 1 ml of the organic solvent.

According to an embodiment of the present invention, the functional inorganic powder may provide a dielectric or insulating function, and include one or more selected from the group consisting of Mg oxide, Fe oxide, Zn oxide, Mo oxide, W oxide, Ca oxide, K oxide, Na oxide, Si oxide, Ge oxide, Mg nitride, Fe nitride, Zn nitride, Mo nitride, W nitride, Ca nitride, K nitride, Na nitride, Si nitride, Ge nitride, and a-IGZO.

According to an embodiment of the present invention, the functional inorganic powder may provide a dielectric or insulating function, and a volume fraction of the functional inorganic powder with respect to a total volume of the paste may range from 0.05 to 0.08.

According to an embodiment of the present invention, the functional inorganic powder may provide a dielectric or insulating function, and the paste may include 0.1 g to 2 g of the functional inorganic powder, 0.1 ml to 1 ml of the humectant, and 0.05 g to 1 g of the matrix polymer, per 1 ml of the organic solvent.

According to an embodiment of the present invention, the humectant may include one or more selected from the group consisting of tetraglycol (TG), ethylene glycol, and N-methyl-2-pyrrolidone (NMP).

According to an embodiment of the present invention, the matrix polymer may include one or more selected from the group consisting of polycaprolactone (PCL), silk fibroin, sodium carboxymethyl cellulose (Na-CMC), polyvinyl alcohol (PVA), polyvinylpyrrolidone (PVP), poly lactic-co-glycolic acid (PLGA), polylactic acid (PLA), polyglycerol sebacate (PGS), and polybutylene adipate terephthalate (PBAT).

According to an embodiment of the present invention, the organic solvent may include one or more selected from the group consisting of tetrahydrofuran (THF), dichloromethane (DCM), chloroform, dimethylformamide (DMF), acetone, and ethyl acetate.

According to an embodiment of the present invention, the paste may have a viscosity ranging from 50 Pa·s to 1000 Pa·s at a shear rate ranging from 1 s⁻¹ to 100 /s⁻¹, and have a yield shear stress ranging from 10² Pa to 10³ Pa at a shear strain ranging from 0.01% to 10%.

According to an embodiment of the present invention, at least one of the functional inorganic powder, the humectant, the matrix polymer, and the organic solvent may be made of a biodegradable material that is decomposed in a human body.

According to an embodiment of the present invention, a method of producing a biodegradable electronic device by using a paste for producing biodegradable electroceuticals may include providing the paste including at least one of conductive, semiconductive, dielectric, and insulating properties, forming an electronic device structure by using the paste, and forming a biodegradable electronic device by providing conductive properties by sintering the electronic device structure.

According to an embodiment of the present invention, the providing of the paste may include forming the paste, by mixing a functional inorganic powder providing conductive, semiconductive, dielectric, or insulating properties, a humectant, a matrix polymer, and an organic solvent at the above-mentioned fractions.

According to an embodiment of the present invention, the forming of the electronic device structure may be performed by three-dimensionally (3D) printing the paste.

According to an embodiment of the present invention, the forming of the biodegradable electronic device may be performed through sintering based on heat treatment, light irradiation, chemical treatment, or electrochemical treatment.

According to an embodiment of the present invention, a biodegradable electronic device formed using the above-described method may include at least one of a diode, a capacitor, an inductor, a resistor, a transistor, an electrode, a rectifier, a switch, a memory, a condenser, and a vibrator.

According to an aspect of the present invention, there is provided a biodegradable electroceutical capable of integrating electronic circuits to implement light, thin, short, and small characteristics and of being decomposed in the human body after a certain period of time so as not to require additional surgery, and a method of producing the same.

According to an embodiment of the present invention, the biodegradable electroceutical may include a plurality of material layers including at least one of an insulator region, a conductor region, and a semiconductor region, and stacked on one another, one or more electronic devices configured by a combination of the plurality of material layers, and a hollow part provided in middle of the plurality of material layers to insert a nerve cell thereinto.

According to an embodiment of the present invention, the electronic devices may be configured over the plurality of material layers in a vertical direction.

According to an embodiment of the present invention, the electronic devices may include at least one of a diode, a capacitor, an inductor, a resistor, a transistor, an electrode, a rectifier, a switch, a memory, a condenser, and a vibrator.

According to an embodiment of the present invention, the material layers may include a first layer including a first conductor region, a second layer including a semiconductor region, and a third layer including a second conductor region, the first to third layers may be sequentially stacked on one another, and the first conductor region, the semiconductor region, and the second conductor region may be vertically aligned to configure a diode as the electronic device.

According to an embodiment of the present invention, the material layers may include a first layer including a first conductor region, a second layer including an insulator region, and a third layer including a second conductor region, the first to third layers may be sequentially stacked on one another, and the first conductor region, the insulator region, and the second conductor region may be vertically aligned to configure a capacitor as the electronic device.

According to an embodiment of the present invention, the material layers may include a first layer including a first conductor region, a second layer including a first insulator region, a third layer including a second conductor region, a fourth layer including a second insulator region, a fifth layer including a third conductor region, a sixth layer including a third insulator region, and a seventh layer including a fourth conductor region, the first to seventh layers may be sequentially stacked on one another, the first conductor region may be electrically connected to the third conductor region, the second conductor region may be electrically connected to the fourth conductor region, the first conductor region, the first insulator region, and the second conductor region may be vertically aligned to configure a first capacitor, the second conductor region, the second insulator region, and the third conductor region may be vertically aligned to configure a second capacitor, and the third conductor region, the third insulator region, and the fourth conductor region may be vertically aligned to configure a third capacitor.

According to an embodiment of the present invention, the first and second capacitors or the second and third capacitors may be alternately engaged with each other.

According to an embodiment of the present invention, the material layers may include a first layer including a first conductor region, and a first insulator region disposed not to connect both ends of the first conductor region, a second layer including a second conductor region in contact with an end of the first conductor region, and a second insulator region disposed to cover and insulate a remaining portion of the first conductor region, and a third layer including a third conductor region in contact with an end of the second conductor region, a third insulator region disposed not to connect both ends of the first conductor region, the first to third layers may be sequentially stacked on one another, and the first to third conductor regions may be vertically disposed to configure an inductor as the electronic device.

According to an embodiment of the present invention, the first to third conductor regions may be wound around the hollow part in a same direction.

According to an embodiment of the present invention, the electronic devices may include a diode, a capacitor, and an inductor, and the capacitor and the inductor may be connected in parallel to each other and connected in series to the diode.

According to an embodiment of the present invention, the capacitor and the inductor may be insulated from each other by the insulator region formed in the plurality of material layers.

According to an embodiment of the present invention, the biodegradable electroceutical may further include an upper electrode electrically connecting an uppermost side of the capacitor to an uppermost side of the inductor.

According to an embodiment of the present invention, the biodegradable electroceutical may further include a lower electrode electrically connecting lowermost sides of the diode.

According to an embodiment of the present invention, at least one of the insulator region, the conductor region, and the semiconductor region may include a biodegradable metal material.

According to an embodiment of the present invention, the conductor region may include magnesium (Mg), iron (Fe), zinc (Zn), molybdenum (Mo), tungsten (W), calcium (Ca), potassium (K), sodium (Na), silicon (Si), amorphous indium gallium zinc oxide (a-IGZO), germanium (Ge), or an alloy thereof.

According to an embodiment of the present invention, a method of producing the above-described biodegradable electroceutical may include providing an electronic circuit including one or more electronic circuit elements, designing a three-dimensional (3D) electroceutical design structure, based on the electronic circuit, designing a plurality of design layers by disassembling the 3D electroceutical design structure into single layers, stacking a plurality of material layers on one another, based on the design layers, and bonding the plurality of material layers to each other to form a biodegradable electroceutical in which electronic devices corresponding to the electronic circuit elements are formed and disposed.

According to an embodiment of the present invention, the biodegradable electroceutical may include a hollow part provided in middle of the plurality of material layers to insert a nerve cell thereinto.

According to an embodiment of the present invention, the stacking of the plurality of material layers on one another may be performed by discharging at least one material from among a conductor, an insulator, and a semiconductor by using a 3D printer.

According to an embodiment of the present invention, the stacking of the plurality of material layers on one another may be performed by forming, on a previously formed material layer, another material layer by discharging at least one material from among a conductor, an insulator, and a semiconductor directly on the previously formed material layer by using the 3D printer.

According to an embodiment of the present invention, the bonding of the plurality of material layers to each other may be performed using at least one of heat treatment, light irradiation, chemical treatment, and electrochemical treatment.

### Advantageous Effects

According to the present invention, a paste for producing biodegradable electroceuticals may simply and easily produce a biodegradable electronic device and a biodegradable electroceutical by forming a biodegradable structure by three-dimensionally (3D) printing a paste providing conductive, semiconductive, dielectric, or insulating properties, and by providing conductive properties by sintering the biodegradable structure.

The biodegradable electroceutical formed using the paste may overcome the limitations of existing implantable electroceuticals. The existing implantable electroceuticals have limitations in that, for example, removal from the human body after use is required due to the use of a permanent material, complicated wire connections are required between electronic devices, electronic devices are required to be produced according to a complicated shape in the human body, and efficient use of a body space is required to place electrodes and devices. Therefore, the biodegradable electroceutical according to the present invention may be made of a biodegradable material to induce natural decomposition after use, and be produced using a 3D printer to simplify the shape, configuration, and production method.

The above-described effects of the present invention are merely examples, and the scope of the present invention is not limited thereto.

### DESCRIPTION OF THE DRAWINGS

FIG. 1 is a schematic view of a biodegradable electroceutical formed using a paste for producing biodegradable electroceuticals, according to an embodiment of the present invention.

FIG. 2 is a graph showing a viscosity based on a shear rate of a paste for producing biodegradable electroceuticals, according to an embodiment of the present invention.

FIG. 3 is a graph showing an elastic modulus based on a shear stress of a paste for producing biodegradable electroceuticals, according to an embodiment of the present invention.

FIG. 4 is a schematic view showing a principle of sintering a conductive structure formed using a paste for producing biodegradable electroceuticals, according to an embodiment of the present invention.

FIG. 5 includes scanning electron microscopic (SEM) images before and after sintering a conductive structure formed using a paste for producing biodegradable electroceuticals, according to an embodiment of the present invention.

FIG. 6 is a graph showing a change in resistance based on a sintering time of a conductive structure formed using a paste for producing biodegradable electroceuticals, according to an embodiment of the present invention.

FIG. 7 includes microscopic images of linear structures formed using a paste for producing biodegradable electroceuticals, according to an embodiment of the present invention.

FIG. 8 is a flowchart of a method of producing a biodegradable electronic device by using a paste for producing biodegradable electroceuticals, according to an embodiment of the present invention.

FIG. 9 includes photographic images of various biodegradable electronic devices formed using a paste for producing biodegradable electroceuticals, according to an embodiment of the present invention.

FIG. 10 is a graph showing a resistance value based on a length of a resistor formed as a biodegradable electronic device by using a paste for producing biodegradable electroceuticals, according to an embodiment of the present invention.

FIG. 11 is a graph showing a capacitance value based on the number of layers of a capacitor formed as a biodegradable electronic device by using a paste for producing biodegradable electroceuticals, according to an embodiment of the present invention.

FIG. 12 is a graph showing an inductance value based on the number of layers of an inductor formed as a biodegradable electronic device by using a paste for producing biodegradable electroceuticals, according to an embodiment of the present invention.

FIG. 13 is a graph showing rectification characteristics of a diode formed as a biodegradable electronic device by using a paste for producing biodegradable electroceuticals, according to an embodiment of the present invention.

FIG. 14 is a photographic image of a transistor formed as a biodegradable electronic device by using a paste for producing biodegradable electroceuticals, according to an embodiment of the present invention.

FIG. 15 is a graph showing a change in drain current based on a drain voltage of a transistor formed as a biodegradable electronic device by using a paste for producing biodegradable electroceuticals, according to an embodiment of the present invention.

FIG. 16 is a schematic view of a three-dimensional (3D) printer for producing a biodegradable electronic device or a biodegradable electroceutical by using a paste for producing biodegradable electroceuticals, according to an embodiment of the present invention.

FIG. 17 includes photographic images of stacked structures formed using a 3D printer and a paste for producing biodegradable electroceuticals, according to an embodiment of the present invention.

FIG. 18 includes photographic images of 3D biodegradable electroceuticals formed using a 3D printer and a paste for producing biodegradable electroceuticals, according to an embodiment of the present invention.

FIG. 19 is a flowchart of a method of producing a biodegradable electroceutical, according to an embodiment of the present invention.

FIG. 20 is a circuit diagram of an electronic circuit of a biodegradable electroceutical, according to an embodiment of the present invention.

FIG. 21 is a schematic view of a 3D stacked model for designing a 3D electroceutical design structure of a biodegradable electroceutical, according to an embodiment of the present invention.

FIG. 22 is a schematic view of a biodegradable electroceutical according to an embodiment of the present invention.

FIGS. 23 to 38 show sequential processes of a method of producing a biodegradable electroceutical, according to an embodiment of the present invention.

### MODE OF THE INVENTION

Hereinafter, the present invention will be described in detail by explaining embodiments of the invention with reference to the attached drawings. The invention may, however, be embodied in many different forms and should not be construed as being limited to the embodiments set forth herein, rather, these embodiments are provided so that this disclosure will be thorough and complete, and will fully convey the concept of the invention to one of ordinary skill in the art. Like reference numerals denote like elements throughout. Various elements and regions are schematically illustrated in the drawings. Therefore, the scope of the present invention is not limited by the sizes or distances shown in the attached drawings.

As used herein, a “design layer” refers to a layer on a design drawing, and a “material layer” refers to a real layer implemented in a biodegradable electroceutical. An “electronic circuit element” refers to an electronic device on a circuit diagram, and an “electronic device” refers to a real electronic device implemented in a biodegradable electroceutical.

Unlike general medical drugs made of chemical compounds, electroceuticals refer to medical devices which provide drug-like effects to human bodies by stimulating the central and peripheral nervous systems by using electronic devices for generating electrical signals. The electroceuticals may be applied to all diseases to be treated using electrical stimulation, e.g., intractable chronic diseases such as diabetes, asthma, chronic airway obstruction, arthritis, hypertension, and gastrointestinal disorder. The electroceuticals are classified into a non-wearable type (grade 1), a wearable type (grade 2), and an implantable type (grades 3 and 4). Continued investment is being made in electroceuticals due to increasing approvals and successful cases thereof in USA, Europe, etc., and the global electroceutical market is expected to grow by 85% from $2 billion in 2018 to $3.8 billion in 2026.

The present invention provides a paste for producing biodegradable electroceuticals, the paste being capable of easily and simply producing the electroceuticals, of being three-dimensionally (3D) printed, and of being decomposed in the human body.

The paste according to the present invention may provide conductive, semiconductive, dielectric, or insulating properties depending on a purpose of use. A biodegradable electronic device may be formed by, for example, stacking the paste. The biodegradable electronic device may include, for example, an active device or a passive device, e.g., a diode, a capacitor, an inductor, a resistor, a transistor, an electrode, a rectifier, a switch, a memory, a condenser, or a vibrator, and may form an integrated circuit. The biodegradable electronic device may be activated through post-processing such as heat treatment, sintering, or drying.

By producing a biodegradable electroceutical by using the paste according to the present invention, the biodegradable electronic device may be applied to the medical device industry, e.g., patient-customized implantable electrical nerve stimulators. Furthermore, technology related to the biodegradable electronic device may be utilized in various fields, e.g., the food industry such as food packaging, the smart farm industry such as soil/plant/illuminance sensors, and the fashion and clothing industry.

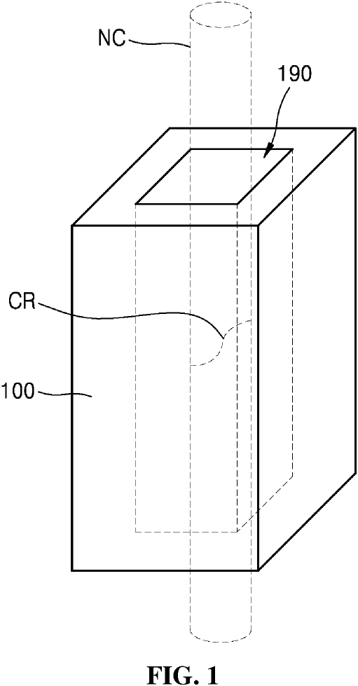

FIG. 1 is a schematic view of a biodegradable electroceutical 100 formed using a paste for producing biodegradable electroceuticals, according to an embodiment of the present invention.

Referring to FIG. 1, the biodegradable electroceutical 100 includes a hollow part 190 therein. A target nerve cell NC to be treated may be inserted into the hollow part 190. For example, nodes of the target nerve cell NC in a severed or damaged target region CR may be separately inserted from above and below the hollow part 190 and connected to each other. Then, biodegradable electronic devices included in the biodegradable electroceutical 100 may transmit electrical signals to the target nerve cell NC to reconstruct and treat the target nerve cell NC. The biodegradable electroceutical 100 may be made of a biodegradable material, and thus be decomposed and absorbed in the human body after treatment or after a certain period of time, thereby not requiring additional surgery for removal.

### Biodegradable Conductive Paste

A paste for producing biodegradable electroceuticals, according to an embodiment of the present invention, includes a functional inorganic powder, a humectant, a matrix polymer, and an organic solvent. The paste includes a mixture of the above-mentioned materials. The paste may be implemented in various fluidic forms, e.g., a liquid, a suspension, a sol, or a gel.

At least one of the functional inorganic powder, the humectant, the matrix polymer, and the organic solvent may be made of a biodegradable material that is decomposed in the human body. The biodegradable material refers to a material that may be absorbed into the human body and is harmless after absorption.

The functional inorganic powder may provide a different function, e.g., conductive, semiconductive, dielectric, or insulating properties, depending on a material included therein.

When the functional inorganic powder provides a conductive function, the functional inorganic powder may include a conductive material. The functional inorganic powder may include one or more selected from the group consisting of, for example, magnesium (Mg), iron (Fe), zinc (Zn), molybdenum (Mo), tungsten (W), calcium (Ca), potassium (K), sodium (Na), silicon (Si), amorphous indium gallium zinc oxide (a-IGZO), germanium (Ge), and alloys thereof.

In the paste, when the functional inorganic powder provides a conductive function, a volume fraction of the functional inorganic powder with respect to a total volume of the paste may range, for example, from 0.35 to 0.41. The volume fraction of the functional inorganic powder may vary depending on a diameter of the functional inorganic powder and, for example, the above-mentioned volume fraction may be obtained when the conductive functional inorganic powder has, for example, a diameter less than or equal to 10 µm, and more specifically, a diameter ranging from 0.5 µm to 10 µm.

In addition, when the functional inorganic powder provides a conductive function, the paste may include, for example, 1 g to 15 g of the functional inorganic powder, 0.1 ml to 1 ml of the humectant, and 0.05 g to 1 g of the matrix polymer, per 1 ml of the organic solvent.

When the functional inorganic powder provides a semiconductive function, the functional inorganic powder may include a semiconductive material. The functional inorganic powder may include one or more selected from the group consisting of, for example, Si, Ge, zinc oxide (ZnO), and aluminum-doped zinc oxide (AZO). When the functional inorganic powder provides a semiconductive function, the functional inorganic powder may have p-type or n-type characteristics through doping.

In the paste, when the functional inorganic powder provides a semiconductive function, a volume fraction of the functional inorganic powder with respect to a total volume of the paste may range, for example, from 0.17 to 0.23. The volume fraction of the functional inorganic powder may vary depending on a diameter of the functional inorganic powder and, for example, the above-mentioned volume fraction may be obtained when the semiconductive functional inorganic powder has, for example, a diameter less than or equal to 400 nm, and more particularly, a diameter ranging from 200 nm to 400 nm.

In addition, when the functional inorganic powder provides a semiconductive function, the paste may include, for example, 1 g to 5 g of the functional inorganic powder, 0.1 ml to 1 ml of the humectant, and 0.05 g to 1 g of the matrix polymer, per 1 ml of the organic solvent.

When the functional inorganic powder provides a dielectric or insulating function, the functional inorganic powder may include a dielectric or insulating material. The functional inorganic powder may include one or more selected from the group consisting of, for example, Mg oxide, Fe oxide, Zn oxide, Mo oxide, W oxide, Ca oxide, K oxide, Na oxide, Si oxide, Ge oxide, Mg nitride, Fe nitride, Zn nitride, Mo nitride, W nitride, Ca nitride, K nitride, Na nitride, Si nitride, Ge nitride, and a-IGZO. For example, the functional inorganic powder may include SiO₂, MgO, or Si₃N₄.

In the paste, when the functional inorganic powder provides a dielectric or insulating function, a volume fraction of the functional inorganic powder with respect to a total volume of the paste may range, for example, from 0.05 to 0.08. The volume fraction of the functional inorganic powder may vary depending on a diameter of the functional inorganic powder and, for example, the above-mentioned volume fraction may be obtained when the dielectric or insulating functional inorganic powder has, for example, a diameter less than or equal to 40 nm, and more specifically, a diameter ranging from 30 nm to 40 nm.

In addition, when the functional inorganic powder provides a dielectric or insulating function, the paste may include, for example, 0.1 g to 2 g of the functional inorganic powder, 0.1 ml to 1 ml of the humectant, and 0.05 g to 1 g of the matrix polymer, per 1 ml of the organic solvent.

The humectant may perform a function of suppressing rapid evaporation of the organic solvent. For example, when the humectant is not included, during a 3D printing operation, the organic solvent may rapidly evaporate and thus the paste may not be easily printed. After the 3D printing operation, the organic solvent may rapidly evaporate and thus a printed solid final structure may experience a severe volume change. Therefore, by including the humectant, undesired solidification of the paste may be prevented and the printed structure may be maintained in shape without being damaged.

The humectant may include one or more selected from the group consisting of, for example, tetraglycol (TG), ethylene glycol, and N-methyl-2-pyrrolidone (NMP).

The matrix polymer may function as a matrix material after the paste is changed into a solid material. The matrix polymer may have stable characteristics against heating, light irradiation, or chemical treatment.

The matrix polymer may include one or more selected from the group consisting of, for example, polycaprolactone (PCL), silk fibroin, sodium carboxymethyl cellulose (Na-CMC), polyvinyl alcohol (PVA), polyvinylpyrrolidone (PVP), poly lactic-co-glycolic acid (PLGA), polylactic acid (PLA), polyglycerol sebacate (PGS), and polybutylene adipate terephthalate (PBAT).

The organic solvent may perform a solvent function for dissolving or dispersing the other components of the paste, and also perform a function for providing fluidity to the paste. The organic solvent may include a liquid material. The organic solvent may be removed when the paste is changed to a solid phase to from a biodegradable electronic device, and thus have volatility or have a lower evaporation temperature compared to the other components. The organic solvent may be easily removed through heating, light irradiation, or chemical treatment.

The organic solvent may include one or more selected from the group consisting of, for example, tetrahydrofuran (THF), dichloromethane (DCM), chloroform, dimethylformamide (DMF), acetone, and ethyl acetate.

Even when pastes for producing biodegradable electroceuticals have different functions such as conductive, semiconductive, dielectric, and insulating properties, the pastes may be discharged from one 3D printer to configure one biodegradable electronic device, and thus have similar viscosities and elastic moduli. The viscosity and the elastic modulus of the paste may vary depending on the volume fraction of the functional inorganic powder.

The viscosity and the elastic modulus of the paste based on a shear rate will now be described. In the following description, Zn powder was used as conductive functional inorganic powder, AZO powder was used as semiconductive functional inorganic powder, and SiO₂/MgO powder was used as dielectric/insulating functional inorganic powder. However, the above-mentioned materials are merely examples and the scope of the present invention is not limited thereto.

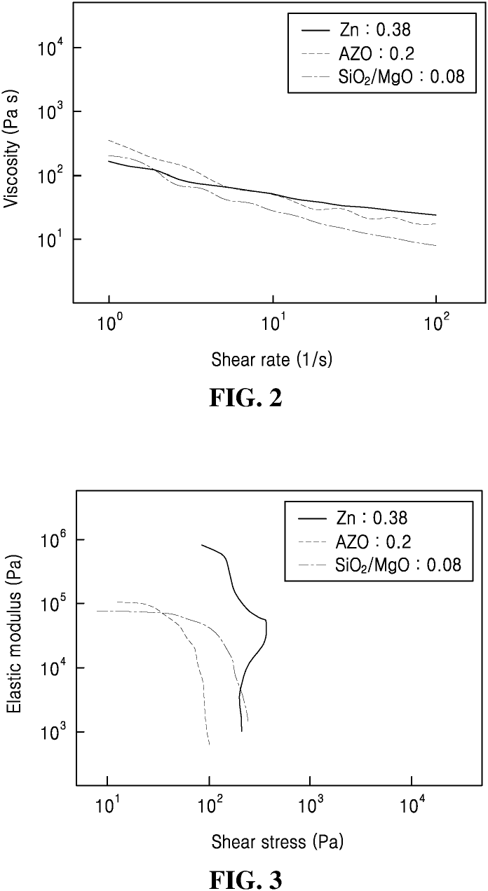

For reference, tests for the viscosity and the elastic modulus, which are described below, were performed at a volume fraction of the Zn powder ranging from 0.35 to 0.41, a volume fraction of the AZO powder ranging from 0.17 to 0.23, and a volume fraction of the SiO₂/MgO powder ranging from 0.05 to 0.08. It merely means that volume fractions shown in FIGS. 2 and 3 may be selected as optimal volume fractions in the above-mentioned ranges, and other volume fractions in the above-mentioned ranges may also be selected for the paste. The above-mentioned volume fractions are merely examples and the scope of the present invention is not limited thereto.

FIG. 2 is a graph showing a viscosity based on a shear rate of a paste for producing biodegradable electroceuticals, according to an embodiment of the present invention.

Referring to FIG. 2, at a shear rate ranging from 1 s⁻¹ to 100/s⁻¹, when a volume fraction of the Zn powder is 0.38, a volume fraction of the AZO powder is 0.2, and a volume fraction of the SiO₂/MgO powder is 0.08 with respect to a total volume of the paste, three types of pastes having different functions exhibit almost similar behaviors of viscosities.

According to the above result, the paste may have, for example, a viscosity ranging from 50 Pa·s to 1000 Pa·s at a shear rate ranging from 1 s⁻¹ to 100/s⁻¹. Specifically, the paste may have, for example, a viscosity ranging from 50 Pa·s to 1000 Pa·s at a shear rate of 10 s⁻¹.

FIG. 3 is a graph showing an elastic modulus based on a shear stress of a paste for producing biodegradable electroceuticals, according to an embodiment of the present invention.

Referring to FIG. 3, at a shear stress ranging from 1 Pa to 1000 Pa, when a volume fraction of the Zn powder is 0.38, a volume fraction of the AZO powder is 0.2, and a volume fraction of the SiO₂/MgO powder is 0.08, three types of pastes having different functions exhibit almost similar behaviors of elastic moduli. The shear stress was measured after applying a shear strain ranging from 0.01% to 10%, and the elastic moduli were calculated based on the relationship between the shear strain and the shear stress.

According to the above result, the paste may have, for example, an elastic modulus ranging from 10² Pa to 10⁶ Pa at a shear stress ranging from 1 Pa to 1000 Pa. The paste may have, for example, an elastic modulus ranging from 10² Pa to 10⁶ Pa at a shear strain ranging from 0.01% to 10%.

In the elastic modulus graph, a yield shear stress may be calculated at a shear stress point where the elastic modulus is rapidly reduced. Therefore, the paste may have a yield shear stress ranging from 10² Pa to 10³ Pa at a shear strain ranging from 0.01% to 10%.

According to the above result, an appropriate fraction of the functional inorganic powder may be set in such a manner that the paste may be used for 3D printing or the like to appropriately produce a biodegradable electronic device or a biodegradable electroceutical. Fractions of the other components may also be set. The fractions of the other components are as mentioned above.

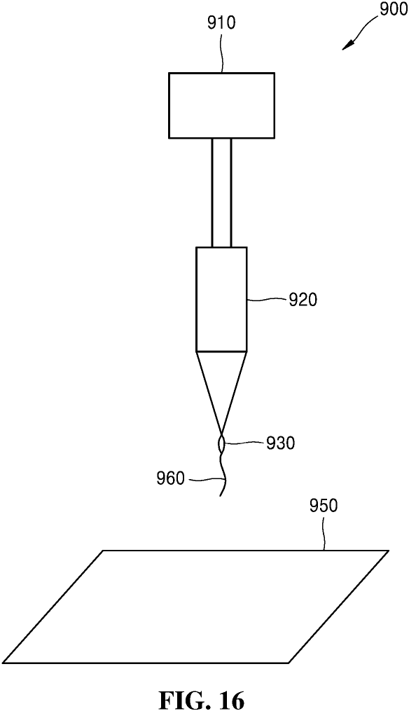

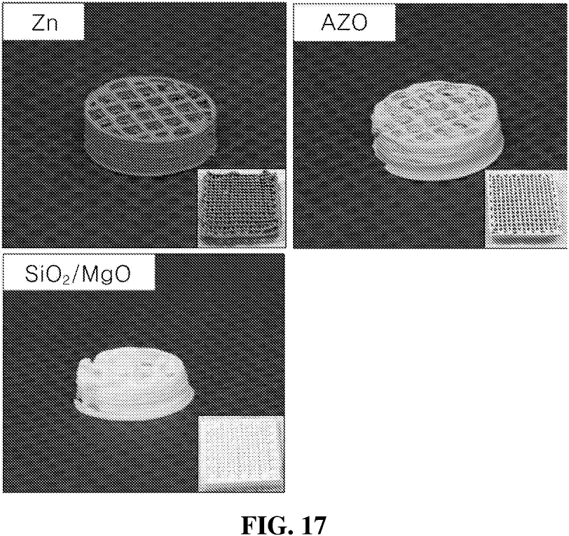

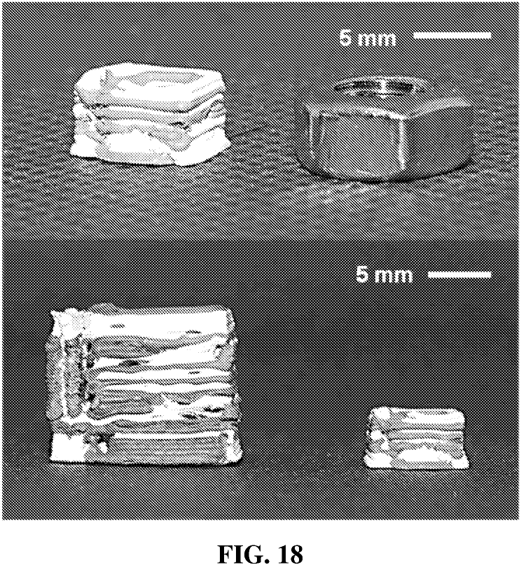

The paste may form various biodegradable electronic devices or biodegradable electroceuticals. For example, the paste may form a biodegradable electronic device by using a 3D printer described below with reference to FIG. 16.

In order for the paste to form a biodegradable electronic device or a biodegradable electroceutical, an electrical function needs to be activated in some cases. That is, when the paste has conductive or semiconductive properties, electrically conductive properties needs to be activated.

In the paste according to the present invention, when a conductive function is required, because a low-conductive matrix polymer is mixed with conductive powder, the conductive properties are reduced compared to a pure conductor. Therefore, the conductive powder needs to be sintered to increase the conductive properties. The sintering is also required when a semiconductive function is performed.

The sintering may use various methods, e.g., an optical, thermal, or chemical method. Electrochemical sintering will now be described as an example.

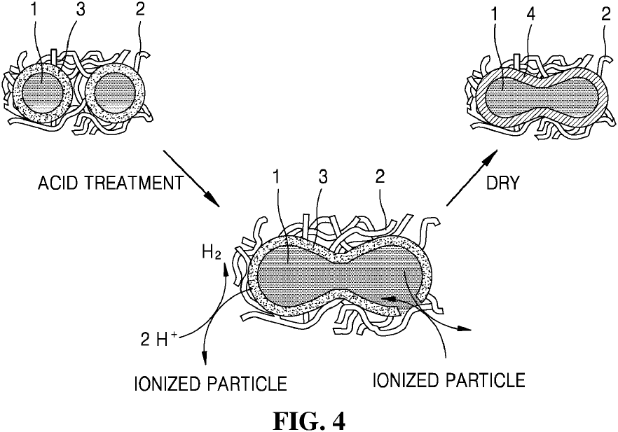

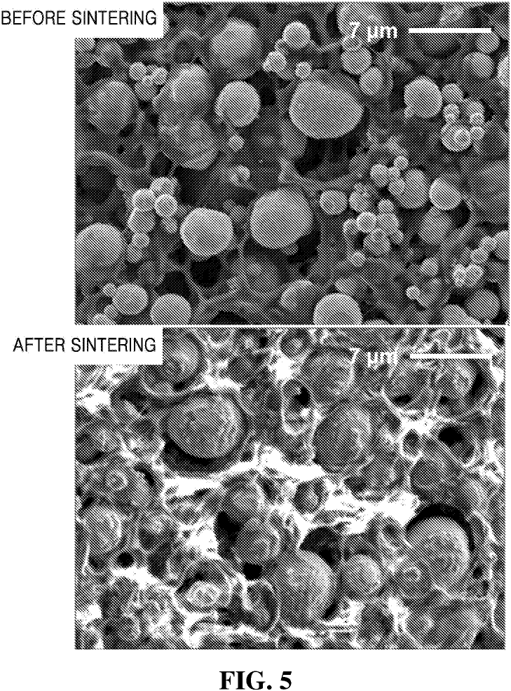

FIG. 4 is a schematic view showing a principle of sintering a conductive structure formed using a paste for producing biodegradable electroceuticals, according to an embodiment of the present invention.

Referring to FIG. 4, a conductive structure may be formed using the paste, and then an organic solvent and a humectant included in the paste may be removed through drying. Therefore, only a conductive functional inorganic powder 1 and a matrix polymer 2 are shown. An initial surface layer 3 may be formed on the surface of the functional inorganic powder 1.

When the above structure is treated with acid such as acetic acid, the initial surface layer 3 is bonded to and removed with hydrogen ions (H⁺) included in the acid, and the functional inorganic powder 1 is exposed. In addition, portions of the inorganic powder 1 may be ionized and discharged outside or be reduced again and attached to the inorganic powder 1. The exposed particles of the functional inorganic powder 1 may be connected and aggregated with each other. Then, when dried, a final surface layer 4 is formed on the surface of the aggregated particles of the functional inorganic powder 1. As such, the functional inorganic powder 1 may form a network of interconnected particles, and conductive properties may be increased.

Herein, each of the initial and final surface layers 3 and 4 may be configured, for example, as an oxide layer or a hydroxide layer, and more specifically, as a zinc oxide (ZnO) layer or a zinc hydroxide (Zn(OH)₂) layer.

The acid treatment may be performed using an acidic solution including various acidic materials, e.g., hydrochloric acid, nitric acid, sulfuric acid, acetic acid, or phosphoric acid. The acidic solution may include the acidic material and water at a ratio of, for example, 1:10 to 10:1.

The acid treatment may be performed by dipping the structure formed using the paste, in the acidic solution, for example, for 1 minute to 60 minutes, and more specifically, for 10 minutes, and then drying the structure by using an inert gas such as a nitrogen gas or an argon gas.

### Test Examples of Conductive Paste

Test examples for a better understanding of the present invention will now be described. However, the following test examples are merely for a better understanding of the present invention, and the present invention is not limited thereto.

### Production of Conductive Paste

The conductive paste for producing biodegradable electroceuticals was produced with the following composition.
Functional inorganic powder: 7.2 g Zn powder
Humectant: 0.5 ml TG
Matrix polymer: 0.15 g PCL
Organic solvent: 1 ml THF

The above-mentioned materials were mixed using revolution mixing and rotation mixing to produce a uniformly mixed conductive paste. In the conductive paste, a volume fraction of the Zn powder with respect to a total volume of the paste was about 0.38.

### Production of Semiconductive Paste

The semiconductive paste for producing biodegradable electroceuticals was produced with the following composition.
Functional inorganic powder: 2.3 g AZO powder
Humectant: 0.5 ml TG
Matrix polymer: 0.15 g PCL
Organic solvent: 1 ml THF

The above-mentioned materials were mixed using revolution mixing and rotation mixing to produce a uniformly mixed semiconductive paste. In the semiconductive paste, a volume fraction of the AZO powder with respect to a total volume of the paste was about 0.2.

### Production of Dielectric or Insulating Paste

The dielectric or insulating paste for producing biodegradable electroceuticals was produced with the following composition.
Functional inorganic powder: 0.18 g of SiO₂ powder and 0.269 g of MgO powder
Humectant: 0.5 ml TG
Matrix polymer: 0.15 g PCL
Organic solvent: 1 ml THF

The above-mentioned materials were mixed using revolution mixing and rotation mixing to produce a uniformly mixed dielectric or insulating paste. In the dielectric or insulating paste, a volume fraction of the SiO₂ powder with respect to a total volume of the paste was about 0.04 and a volume fraction of the MgO powder was about 0.04. Therefore, a sum of the volume fractions of the powders is 0.08.

FIG. 5 includes scanning electron microscopic (SEM) images before and after sintering a conductive structure formed using a paste for producing biodegradable electroceuticals, according to an embodiment of the present invention.

Referring to FIG. 5, the paste includes Zn as a functional inorganic powder, and thus may provide a conductive function. A microstructure of the conductive structure formed using the paste is observed before and after sintering.

Before sintering, spherical Zn particles are individually separate and a connection structure between the particles is hardly observed.

On the contrary, after sintering, a network structure shape connecting between the Zn particles is observed. In addition, adjacent particles are closely connected to each other to exhibit a sintered shape other than the original individual spherical shape. The network and the sintered shape may increase conductive properties. The above sintering result may also be applied to a semiconductive structure.

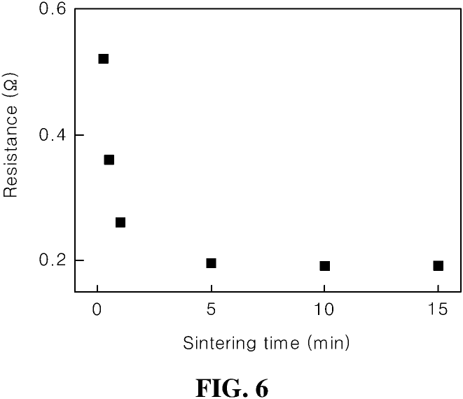

FIG. 6 is a graph showing a change in resistance based on a sintering time of a conductive structure formed using a paste for producing biodegradable electroceuticals, according to an embodiment of the present invention.

Referring to FIG. 6, a result related to the conductive structure of FIG. 5 is shown. Before sintering, the conductive structure exhibits a high resistance value of about 0.55 Ω, and it is analyzed that a high resistance, i.e., a low conductivity, is exhibited because the conductive Zn particles are separate and not electrically connected to each other. After sintering is performed, the resistance value is rapidly reduced. The rapid reduction is seen within initial 1 minute. 0.2 Ω is exhibited at a sintering time of 5 minutes, and the resistance is hardly changed thereafter even when the sintering time is increased. Therefore, the conductive structure may provide an excellent conductive function due to the above-mentioned electrochemical sintering.

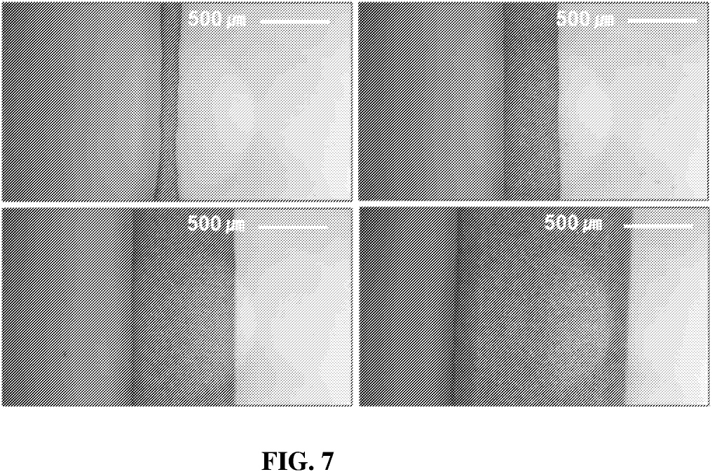

FIG. 7 includes microscopic images of linear structures formed using a paste for producing biodegradable electroceuticals, according to an embodiment of the present invention.

Referring to FIG. 7, various-sized linear structures formed by 3D-printing the paste, which includes Zn as a functional inorganic powder, are shown. The linear structures may have various line widths depending on a nozzle of a 3D printer. The linear structures have, for example, a line width ranging from 150 µm to 1000 µm, and are formed to have a very high uniformity without defects such as breakage, contraction, and expansion of lines in the above-mentioned range.

The above result is equally obtained when the paste has semiconductive, dielectric, or insulating properties. Therefore, the paste may be applied to 3D printing to produce a biodegradable electronic device and a biodegradable electroceutical.

### Biodegradable Electronic Device

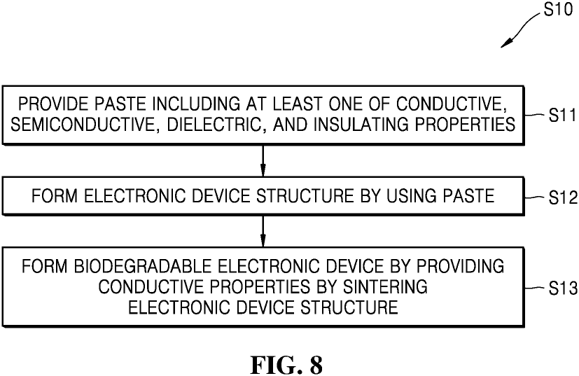

FIG. 8 is a flowchart of a method S10 of producing a biodegradable electronic device by using a paste for producing biodegradable electroceuticals, according to an embodiment of the present invention.

Referring to FIG. 8, the method S10 is a method of producing a biodegradable electronic device by using a paste for producing biodegradable electroceuticals, and includes providing the paste including at least one of conductive, semiconductive, dielectric, and insulating properties (S11), forming an electronic device structure by using the paste (S12), and forming a biodegradable electronic device by providing conductive properties by sintering the electronic device structure (S13).

The providing of the paste (S11) includes forming the paste having conductive, semiconductive, dielectric, or insulating properties, according to the above-described method. The paste may be formed by mixing a functional inorganic powder providing conductive, semiconductive, dielectric, or insulating properties, a humectant, a matrix polymer, and an organic solvent at the above-mentioned fractions.

The forming of the electronic device structure (S12) may be performed by 3D-printing the paste. Specifically, the electronic device structure may be formed by discharging the paste according to a design of electronic device by using a 3D printer.

The forming of the biodegradable electronic device (S13) may be performed through the above-mentioned electrochemical sintering. However, the electrochemical sintering is merely an example and the sintering may include sintering based on heat treatment, light irradiation, chemical treatment, or electrochemical treatment.

A biodegradable electronic device formed using the above-described method may include at least one of a diode, a capacitor, an inductor, a resistor, a transistor, an electrode, a rectifier, a switch, a memory, a condenser, and a vibrator.

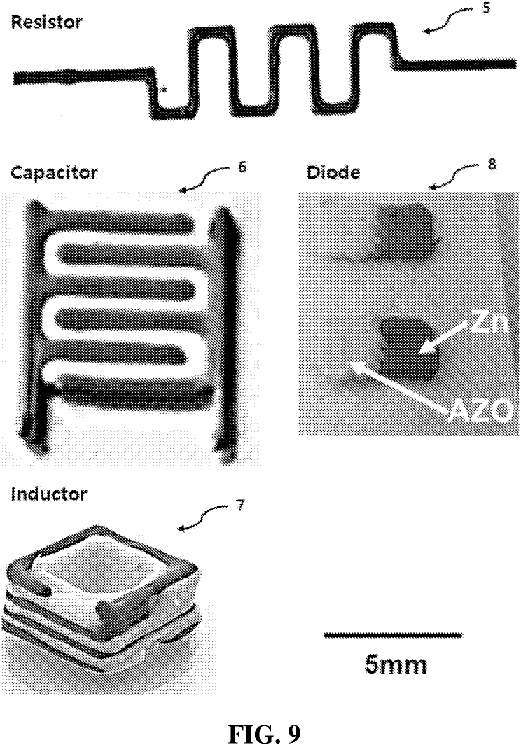

FIG. 9 includes photographic images of various biodegradable electronic devices formed using a paste for producing biodegradable electroceuticals, according to an embodiment of the present invention.

Referring to FIG. 9, various biodegradable electronic devices were formed using the paste. The biodegradable electronic devices may include, for example, a resistor 5, a capacitor 6, an inductor 7, and a diode 8. Structures for the biodegradable electronic devices were formed using 3D printing, and conductive properties were ensured through electrochemical sintering when necessary. When the 3D printing was performed, by alternately discharging a conductive paste, a semiconductive paste, a dielectric paste, and an insulating paste by using a plurality of nozzles, the biodegradable electronic devices were formed in one process.

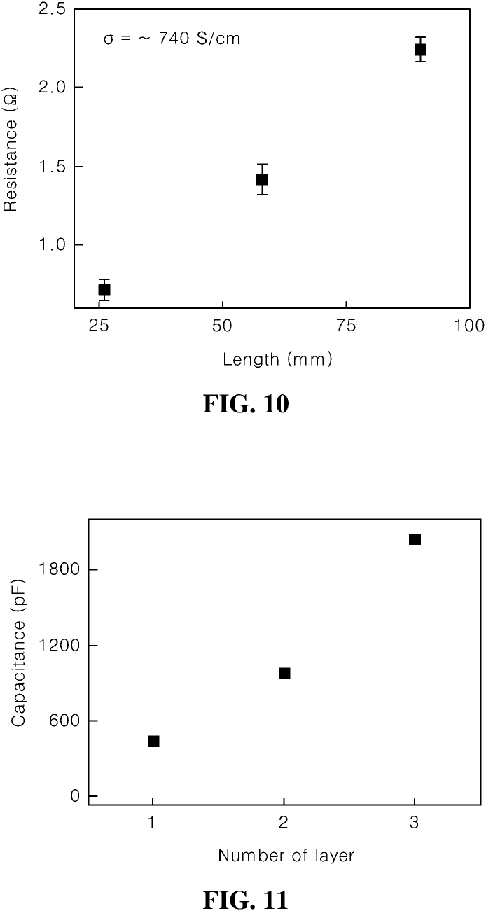

FIG. 10 is a graph showing a resistance value based on a length of a resistor formed as a biodegradable electronic device by using a paste for producing biodegradable electroceuticals, according to an embodiment of the present invention.

Referring to FIG. 10, the resistance value is increased linearly in proportion to the length of the resistor. A conductivity σ of the resistor is about 740 S/m. Therefore, because the resistor may effectively perform a resistor function, the paste may be effectively used to form the resistor.

FIG. 11 is a graph showing a capacitance value based on the number of layers of a capacitor formed as a biodegradable electronic device by using a paste for producing biodegradable electroceuticals, according to an embodiment of the present invention.

Referring to FIG. 11, the capacitance value is increased almost linearly in proportion to the number of layers of the capacitor. Because the capacitor may effectively perform a capacitor function, the paste may be effectively used to form the capacitor.

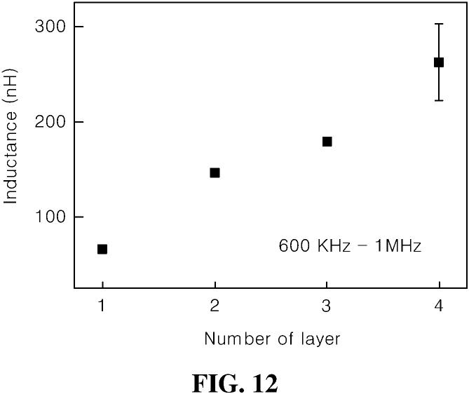

FIG. 12 is a graph showing an inductance value based on the number of layers of an inductor formed as a biodegradable electronic device by using a paste for producing biodegradable electroceuticals, according to an embodiment of the present invention.

Referring to FIG. 12, the inductance value is increased almost linearly in proportion to the number of layers of the inductor. Because the inductor may effectively perform an inductor function, the paste may be effectively used to form the inductor.

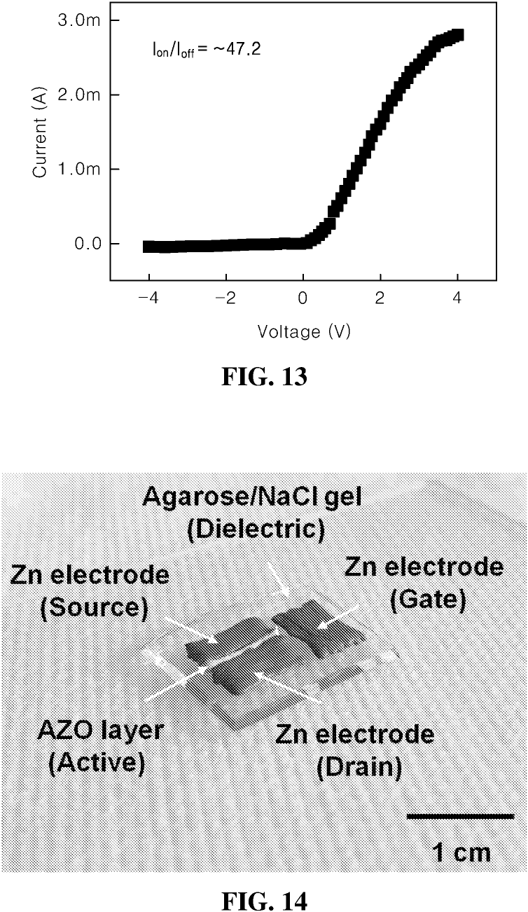

FIG. 13 is a graph showing rectification characteristics of a diode formed as a biodegradable electronic device by using a paste for producing biodegradable electroceuticals, according to an embodiment of the present invention.

Referring to FIG. 13, the diode exhibits a current which is increased linearly in proportion to a voltage at positive voltages. The diode exhibits a current of 0A at negative voltages. An Iₒₙ/I_{off} ratio of the diode is about 47.2. Because the diode may effectively perform a rectification function, the paste may be effectively used to form the diode.

The paste may also be used to implement an active device such as a transistor.

FIG. 14 is a photographic image of a transistor formed as a biodegradable electronic device by using a paste for producing biodegradable electroceuticals, according to an embodiment of the present invention.

Referring to FIG. 14, in the transistor formed as a biodegradable electronic device, a source, a drain, and a gate made of Zn were formed using a conductive paste for producing biodegradable electroceuticals. In addition, an active channel made of AZO was formed using a semiconductive paste for producing biodegradable electroceuticals. The transistor was formed on dielectric agarose/sodium chloride (NaCl) gel which is a biodegradable material.

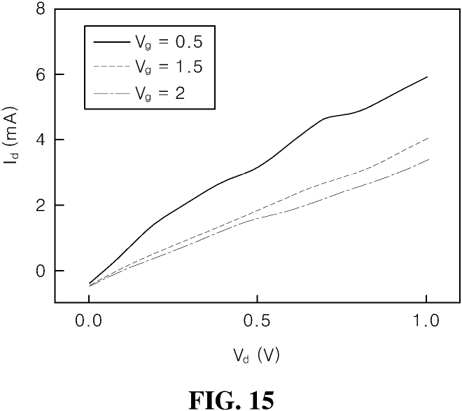

FIG. 15 is a graph showing a change in drain current based on a drain voltage of a transistor formed as a biodegradable electronic device by using a paste for producing biodegradable electroceuticals, according to an embodiment of the present invention.

Referring to FIG. 15, the drain current of the transistor is increased almost linearly in proportion to the drain voltage. The linear increase is equally exhibited at various gate voltages. When the gate voltage is increased, a gradient is reduced. Because the transistor may effectively perform a transistor function, the paste may be effectively used to form the transistor.

A 3D printing method for forming a biodegradable structure such as a biodegradable electronic device or a biodegradable electroceutical by using the paste will now be described.

FIG. 16 is a schematic view of a 3D printer 900 for producing a biodegradable electronic device or a biodegradable electroceutical by using a paste for producing biodegradable electroceuticals, according to an embodiment of the present invention.

Referring to FIG. 16, the 3D printer 900 includes a spinning solution tank 910, a spinning nozzle 920, a spinning nozzle tip 930, and a collector 950.

The 3D printer 900 uses direct ink writing (DIW) to directly form a spinning solution 960 into layers stacked on one another. According to the DIW method, when the spinning solution 960 such as an ink or a paste is pushed using a screw, a piston, or a pressure, the spinning solution 960 is discharged through the spinning nozzle 920. Then, the discharged spinning solution 960 is solidified on the collector 950. After a layer is solidified, another layer may be stacked and solidified thereon to form a 3D structure. In this case, an external stimulus, e.g., ultraviolet light or reaction with calcium chloride (CaCl₂), or the characteristics of the spinning solution 960, e.g., rapid evaporation of a solvent or formation of an oxide layer on the surface, may be used for the solidification. The biodegradable electroceutical according to an embodiment of the present invention may use a method of pushing out the spinning solution 960 with a pressure and solidifying the spinning solution 960 by using the characteristics of the spinning solution 960. However, the above-described method is merely an example and the scope of the present invention is not limited thereto.

The spinning solution tank 910 may store the spinning solution 960. The spinning solution tank 910 may provide the spinning solution 960 to the spinning nozzle 920 by pressing the spinning solution 960 by using an embedded pump (not shown). The spinning nozzle 920 may receive the spinning solution 960 from the spinning solution tank 910 and extrude the spinning solution 960 through the spinning nozzle tip 930 positioned at an end thereof. After the spinning solution 960 is pressed by the pump to fill a nozzle tube, the spinning nozzle tip 930 may extrude the spinning solution 960 based on a voltage applied from a power source 940. The collector 950 is positioned below the spinning nozzle 920 to receive the extruded spinning solution 960.

The above-described relative position between the collector 950 and the spinning nozzle 920 is merely an example, and the scope of the present invention is not limited thereto. For example, a case in which the collector 950 is positioned above the spinning nozzle 920 and the spinning solution 960 is extruded upward from the spinning nozzle 920 is also included in the scope of the present invention. For example, a case in which the collector 950 is positioned side by side with the spinning nozzle 920 and the spinning solution 960 is extruded horizontally from the spinning nozzle 920 is also included in the scope of the present invention. The collector 950 may be positioned side by side with or on the same spatial axis as the spinning nozzle 920.

Conditions for 3D-printing the spinning solution 960 in a pneumatic manner are as follows. The spinning solution 960 needs to be discharged through the spinning nozzle 920 in the form of a filament. Layers formed using the spinning solution 960 need to be easily stacked on one another. The spinning solution 960 needs to have viscoelastic properties to exhibit a shear modulus that is constantly maintained and then reduced when a shear stress applied thereto is increased. The spinning solution 960 needs to have shear thinning properties to exhibit a viscosity that is reduced when a shear rate thereof is increased.

The spinning solution 960 may vary depending on a material to be extruded, and include, for example, materials for configuring the above-described conductor, insulator, and semiconductor regions. The spinning solution 960 may form an ink or a paste by mixing a polymer and conductive particles. Therefore, compared to a pure conductor, conductive properties of a conductor region may be reduced.

To increase the conductive properties of the conductor region, a conductive network of the conductive particles included in the conductor region may be formed through sintering based on light irradiation, heating, or chemical reaction. For example, electrochemical sintering may be used in the present invention. For example, an oxide layer formed on the surface of the conductive particles may be removed using acid, ionized conductive atoms may be reduced again, and a conductive network between the conductive particles may be formed.

FIG. 17 includes photographic images of stacked structures formed using a 3D printer and a paste for producing biodegradable electroceuticals, according to an embodiment of the present invention.

Referring to FIG. 17, a real stacked structure of Zn capable of configuring a conductor region, a real stacked structure of AZO capable of configuring a semiconductor region, and a real stacked structure of SiO₂/MgO capable of configuring an insulator region of a biodegradable electroceutical are shown. The stacked structures are formed by stacking a plurality of layers extruded from a 3D printer. In all cases, it is shown that the stacked structures may be easily formed and freely shaped. For reference, inset images show examples of layers formed by being extruded from a 3D printer.

FIG. 18 includes photographic images of 3D biodegradable electroceuticals formed using a 3D printer and a paste for producing biodegradable electroceuticals, according to an embodiment of the present invention.

Referring to FIG. 18, biodegradable electroceuticals formed by stacking Zn capable of configuring a conductor region, AZO capable of configuring a semiconductor region, and SiO₂ or SiO₂/MgO capable of configuring an insulator region are shown. It is shown that the biodegradable electroceuticals produced according to the present invention may be easily formed by stacking the materials having different properties of a conductor, a semiconductor, and an insulator. It is also shown that the biodegradable electroceuticals may be formed in various sizes, e.g., in diameters ranging from 5 mm to 15 mm. However, the above-mentioned sizes are merely examples and the scope of the present invention is not limited thereto.

### Biodegradable Electroceutical

The configuration of and a method of producing the biodegradable electroceutical 100 of FIG. 1 will now be described as an example. However, the following description is merely an example and the present invention is not limited thereto.

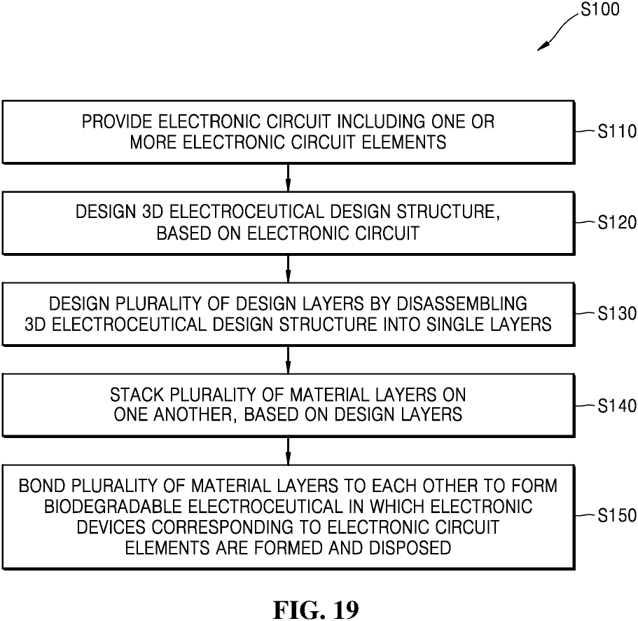

FIG. 19 is a flowchart of a method S100 of producing a biodegradable electroceutical, according to an embodiment of the present invention.

Referring to FIG. 19, the method S100 includes providing an electronic circuit including one or more electronic circuit elements (S110), designing a 3D electroceutical design structure, based on the electronic circuit (S120), designing a plurality of design layers by disassembling the 3D electroceutical design structure into single layers (S130), stacking a plurality of material layers on one another, based on the design layers (S140), and bonding the plurality of material layers to each other to form a biodegradable electroceutical in which electronic devices corresponding to the electronic circuit elements are formed and disposed (S150).

A biodegradable electroceutical may be formed using the method S100 of FIG. 19.

The biodegradable electroceutical may include a plurality of material layers including at least one of an insulator region, a conductor region, and a semiconductor region, and stacked on one another, one or more electronic devices configured by a combination of the plurality of material layers, and a hollow part provided in the middle of the plurality of material layers to insert a nerve cell thereinto.

The electronic devices may be configured over the plurality of material layers in a vertical direction.

The electronic devices may include at least one of a diode, a capacitor, an inductor, a resistor, a transistor, an electrode, a rectifier, a switch, a memory, a condenser, and a vibrator.

The material layers may include a first layer including a first conductor region; a second layer including a semiconductor region; and a third layer including a second conductor region, the first to third layers may be sequentially stacked on one another, and the first conductor region, the semiconductor region, and the second conductor region may be vertically aligned to configure a diode as the electronic device.

The material layers may include a first layer including a first conductor region, a second layer including an insulator region, and a third layer including a second conductor region, the first to third layers may be sequentially stacked on one another, and the first conductor region, the insulator region, and the second conductor region may be vertically aligned to configure a capacitor as the electronic device.

The material layers may include a first layer including a first conductor region; a second layer including a first insulator region; a third layer including a second conductor region; a fourth layer including a second insulator region; a fifth layer including a third conductor region; a sixth layer including a third insulator region; and a seventh layer including a fourth conductor region, the first to seventh layers may be sequentially stacked on one another, the first conductor region may be electrically connected to the third conductor region, the second conductor region may be electrically connected to the fourth conductor region, the first conductor region, the first insulator region, and the second conductor region may be vertically aligned to configure a first capacitor, the second conductor region, the second insulator region, and the third conductor region may be vertically aligned to configure a second capacitor, and the third conductor region, the third insulator region, and the fourth conductor region may be vertically aligned to configure a third capacitor.

The first and second capacitors or the second and third capacitors may be alternately engaged with each other.

The material layers may include a first layer including a first conductor region, and a first insulator region disposed not to connect both ends of the first conductor region; a second layer including a second conductor region in contact with an end of the first conductor region, and a second insulator region disposed to cover and insulate a remaining portion of the first conductor region; and a third layer including a third conductor region in contact with an end of the second conductor region, a third insulator region disposed not to connect both ends of the first conductor region, the first to third layers may be sequentially stacked on one another, and the first to third conductor regions may be vertically disposed to configure an inductor as the electronic device.

The first to third conductor regions may be wound around the hollow part in the same direction.

The electronic devices may include a diode, a capacitor, and an inductor. The capacitor and the inductor may be connected in parallel to each other and connected in series to the diode.

The capacitor and the inductor may be insulated from each other by the insulator region formed in the plurality of material layers.

The biodegradable electroceutical may further include an upper electrode electrically connecting an uppermost side of the capacitor to an uppermost side of the inductor.

The biodegradable electroceutical may further include a lower electrode electrically connecting lowermost sides of the diode.

At least one of the insulator region, the conductor region, and the semiconductor region may include a biodegradable metal material.

The insulator region may include various insulating materials or dielectric materials.

The conductor region may include Mg, Fe, Zn, Mo, W, Ca, K, Na, Si, a-IGZO, Ge, or an alloy thereof.

The semiconductor region may include various semiconductive materials, and the semiconductive material may have p-type or n-type characteristics through doping.

An example of the biodegradable electroceutical 100 formed according to the method S100 of FIG. 19 will now be described.

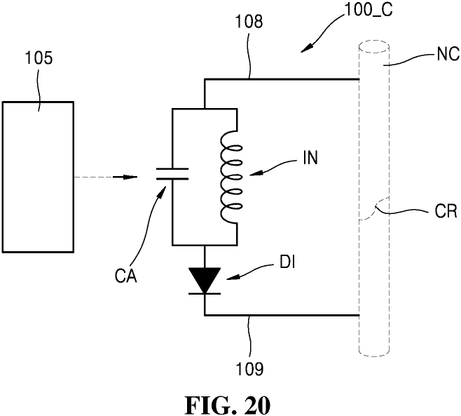

Initially, the providing of the electronic circuit including the one or more electronic circuit elements (S110) of FIG. 19 is performed. As such, a circuit diagram of FIG. 20 may be obtained.

FIG. 20 is a circuit diagram of an electronic circuit 100_C of the biodegradable electroceutical 100, according to an embodiment of the present invention.

Referring to FIG. 20, an example in which the providing of the electronic circuit (S110) is implemented is shown. The electronic circuit 100_C is an electronic circuit for the biodegradable electroceutical 100, and includes a diode DI, a capacitor CA, and an inductor IN as the electronic circuit elements. The electronic circuit elements may include various electronic circuit elements, e.g., at least one of a diode, a capacitor, an inductor, a resistor, a transistor, an electrode, a rectifier, a switch, a memory, a condenser, and a vibrator.

In the electronic circuit 100_C, the capacitor CA and the inductor IN may be connected in parallel to each other and connected in series to the diode DI. Although the diode DI, the capacitor CA, and the inductor IN are illustrated in singular forms, the illustration is merely an example and plural forms thereof are also included in the scope of the present invention. In addition, the present invention is not limited by the number, positions, and types of the electronic circuit elements.

The electronic circuit 100_C may receive power from an external wireless power source 105 in a wireless manner. That is, an LC circuit in the electronic circuit 100_C may be resonated by an induced current from the external wireless power source 105, thereby receiving power.

The electronic circuit 100_C may have a first wire 108 and a second wire 109, and the first and second wires 108 and 109 may be electrically or physically connected to or in contact with the target nerve cell NC. Therefore, the electronic circuit 100_C may generate an electrical signal by receiving power from the external wireless power source 105, and transmit the electrical signal through the first and second wires 108 and 109 to the target nerve cell NC to provide the electrical signal for treatment to the target region CR.

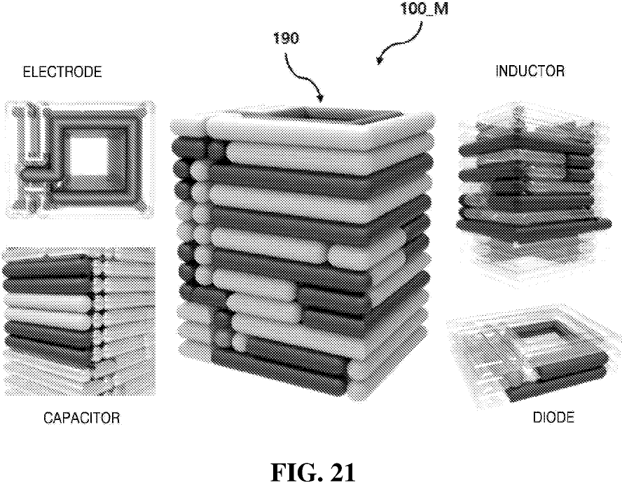

Then, the designing of the 3D electroceutical design structure (S120) is performed based on the electronic circuit of FIG. 20. For example, conditions for designing the 3D electroceutical design structure based on the electronic circuit 100_C are as follows. A resonant frequency is 25 MHz, a capacitor includes at least four layers, an inductor is configured as a coil wound around the hollow part 190 by five turns, and a diode is disposed at a lower side. As such, a 3D stacked model 100_M of FIG. 21 may be obtained.

FIG. 21 is a schematic view of a 3D stacked model 100_M for designing a 3D electroceutical design structure of the biodegradable electroceutical 100, according to an embodiment of the present invention.

Referring to FIG. 21, the 3D stacked model 100_M of the biodegradable electroceutical 100 may be configured by stacking a plurality of planar layers on one another in 3D. The 3D stacked model 100_M may be designed with a configuration in which a diode is disposed at a lower side, a capacitor is disposed on a side surface, an inductor is disposed on another side surface to surround the hollow part 190, and electrodes are disposed at uppermost and lowermost sides.

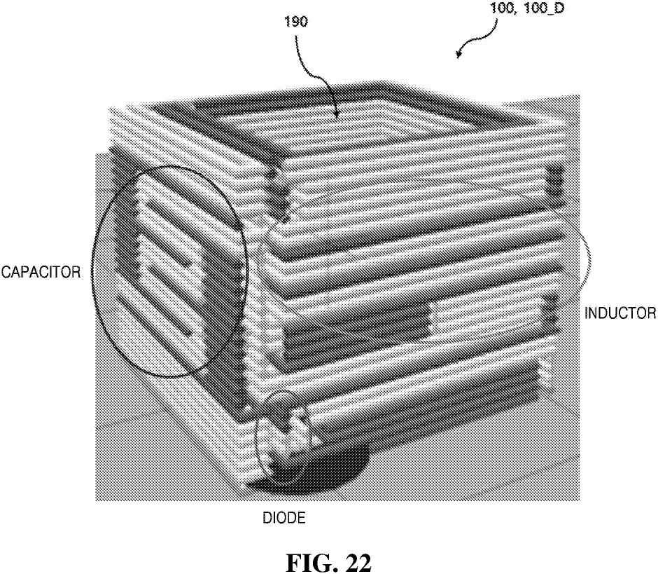

Then, the designing of the plurality of design layers (S130) is performed by disassembling a 3D electroceutical design structure 100_D of FIG. 22 into single layers. Thereafter, the forming of the material layers, based on the design layers (S140) is performed. Then, the stacking of the plurality of material layers on one another to form the biodegradable electroceutical in which electronic devices corresponding to the electronic circuit elements are formed and disposed (S150) is performed. As such, the biodegradable electroceutical 100 of FIG. 22 may be formed.

The forming of the material layers (S140) may be performed using a 3D printer. Specifically, the stacking of the plurality of material layers on one another (S140) may be performed by discharging at least one material from among a conductor, an insulator, and a semiconductor by using a 3D printer. The stacking of the plurality of material layers on one another (S140) may be performed by forming, on a previously formed material layer, another material layer by discharging at least one material from among a conductor, an insulator, and a semiconductor directly on the previously formed material layer by using the 3D printer.

The bonding of the plurality of material layers to each other (S150) may be performed using at least one of heat treatment, light irradiation, chemical treatment, and electrochemical treatment.

FIG. 22 is a schematic view of the biodegradable electroceutical 100 according to an embodiment of the present invention.

Referring to FIG. 22, the illustrated biodegradable electroceutical 100 may also represent the above-described 3D electroceutical design structure 100_D. That is, the following description of the biodegradable electroceutical 100 may also be understood as that of the 3D electroceutical design structure 100_D.

The biodegradable electroceutical 100 may have a configuration in which a plurality of material layers are stacked on one another in 3D. Therefore, when the biodegradable electroceutical 100 is disassembled into single layers, it may be divided into the plurality of material layers. Likewise, the 3D electroceutical design structure 100_D may have a configuration in which a plurality of design layers are stacked on one another in 3D. Therefore, when the 3D electroceutical design structure 100_D is disassembled into single layers, it may be divided into the plurality of design layers.

The biodegradable electroceutical 100 may include the hollow part 190 provided in the middle to insert a nerve cell thereinto. In the 3D electroceutical design structure 100_D of the biodegradable electroceutical 100, a diode is disposed at a lower side, a capacitor (shown in blue) is disposed on a side surface, and an inductor extending to surround the hollow part 190 therein is disposed on another side surface. The diode, the capacitor, and the inductor are formed in a direction perpendicular to a plane formed by the design layer. In this case, each of the diode, the capacitor, and the inductor may be designed to be disposed over a plurality of design layers.

The forming of the material layers may be performed using a 3D printer.

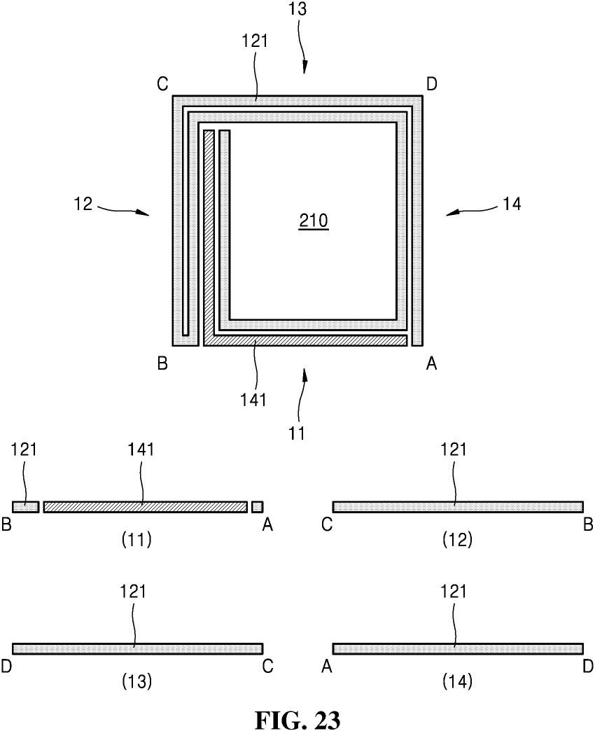

FIGS. 23 to 38 show sequential processes of a method of producing the biodegradable electroceutical 100, according to an embodiment of the present invention.

In FIGS. 23 to 38, an upper side shows a top view and a lower side shows four side views. The following production processes assume an example in which the biodegradable electroceutical 100 is formed using a 3D printer. A “line” mentioned below may be formed by a filament discharged from the 3D printer, and also referred to as a “layer” to indicate a “design layer” or a “material layer” in FIGS. 23 to 38.

Referring to FIG. 23, a first layer 210 is formed. The first layer 210 may be formed by forming a first insulator region 121 and a first conductor region 141.

On a first side surface 11, the first conductor region 141 may extend at an outermost side, and be formed as one or more lines. The first conductor region 141 may extend to be inserted between portions of the first insulator region 121 on a second side surface 12.

On the second side surface 12, the first insulator region 121 may extend at an outermost side, and be formed as one or more lines. The first conductor region 141 may extend between portions of the first insulator region 121. The first conductor region 141 disposed on the second side surface 12 may be formed as one or more lines.

On a third side surface 13, the first insulator region 121 may extend at an outermost side, and be formed as one or more lines.

On a fourth side surface 14, the first insulator region 121 may extend at an outermost side, and be formed as one or more lines.

The first conductor region 141 formed on the first side surface 11 and the first insulator region 121 formed on the second to fourth side surfaces 12 to 14 may form outer walls.

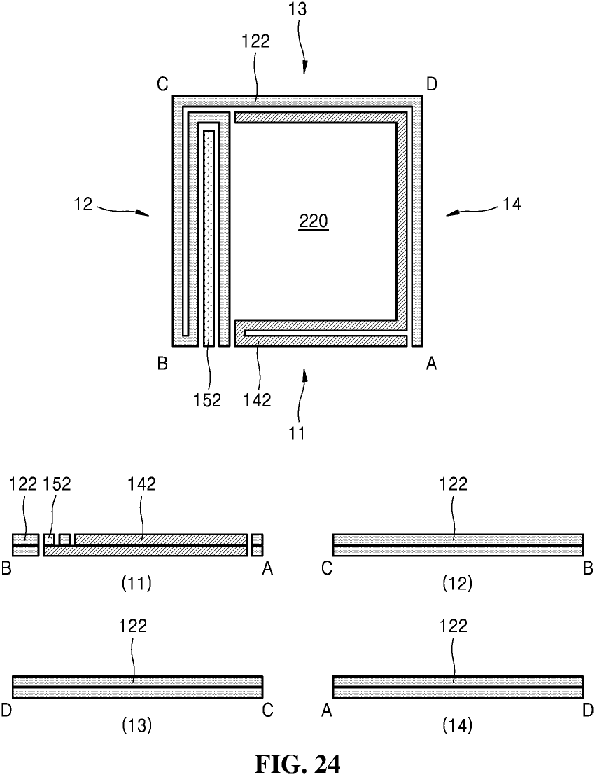

Referring to FIG. 24, a second layer 220 is formed. The second layer 220 may be formed by forming a second insulator region 122, a second conductor region 142, and a semiconductor region 152.

On the first side surface 11, the second conductor region 142 may extend at an outermost side, and be formed as one or more lines.

On the second side surface 12, the second insulator region 122 may extend at an outermost side, and be formed as one or more lines. The semiconductor region 152 may extend between portions of the second insulator region 122. The semiconductor region 152 disposed on the second side surface 12 may be formed as one or more lines.

On the third side surface 13, the second insulator region 122 may extend at an outermost side, and be formed as at least one line. The second conductor region 142 may extend at an inner side of the second insulator region 122, and be formed as one or more lines.

On the fourth side surface 14, the second insulator region 122 may extend at an outermost side, and be formed as at least one line. The second conductor region 142 may extend at an inner side of the second insulator region 122, and be formed as one or more lines.

The second conductor region 142 formed on the first side surface 11 and the second insulator region 122 formed on the second to fourth side surfaces 12 to 14 may form outer walls.

The first conductor region 141 of the first layer 210 and the second conductor region 142 of the second layer 220, which are in contact with each other, may be bonded and electrically connected to each other. The first insulator region 121 of the first layer 210 and the second insulator region 122 of the second layer 220, which are in contact with each other, may be bonded to each other through sintering or the like.

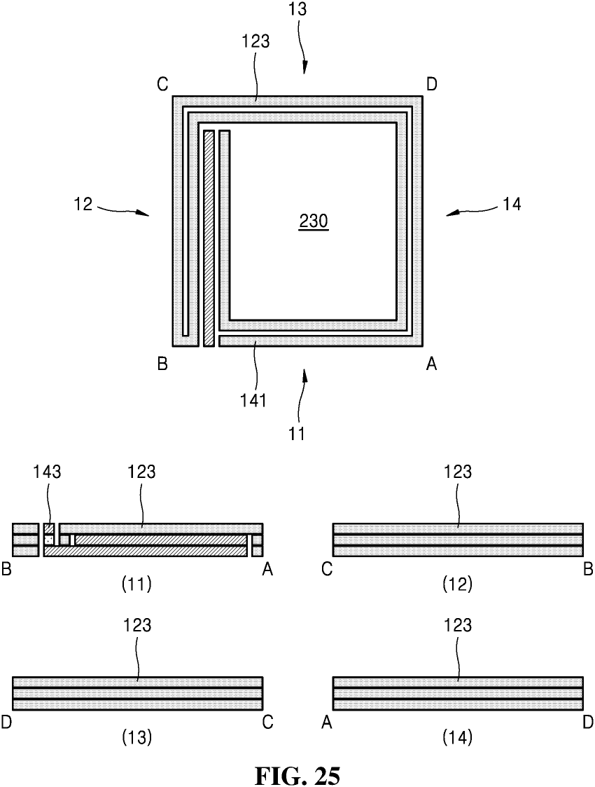

Referring to FIG. 25, a third layer 230 is formed. The third layer 230 may be formed by forming a third insulator region 123 and a third conductor region 143.

On the first side surface 11, the third conductor region 143 may extend at an outermost side, and be formed as one or more lines.

On the second side surface 12, the third insulator region 123 may extend at an outermost side, and be formed as one or more lines. The third conductor region 143 may extend between portions of the third insulator region 123. The third conductor region 143 disposed on the second side surface 12 may be formed as one or more lines. That is, the third conductor region 143 and the third insulator region 123 may be alternately disposed.

On the third side surface 13, the third insulator region 123 may extend at an outermost side, and be formed as one or more lines.

On the fourth side surface 14, the third insulator region 123 may extend at an outermost side, and be formed as one or more lines.

The third insulator region 123 formed on the first to fourth side surfaces 11 to 14 may form outer walls.

The second conductor region 142 of the second layer 220 and the third conductor region 143 of the third layer 230, which are in contact with each other, may be bonded and electrically connected to each other. The second insulator region 122 of the second layer 220 and the third insulator region 123 of the third layer 230, which are in contact with each other, may be bonded to each other through sintering or the like.

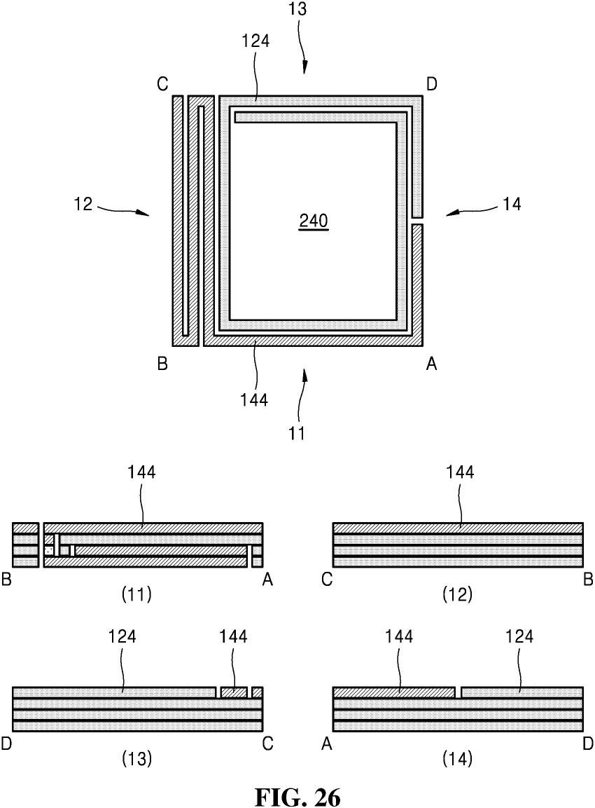

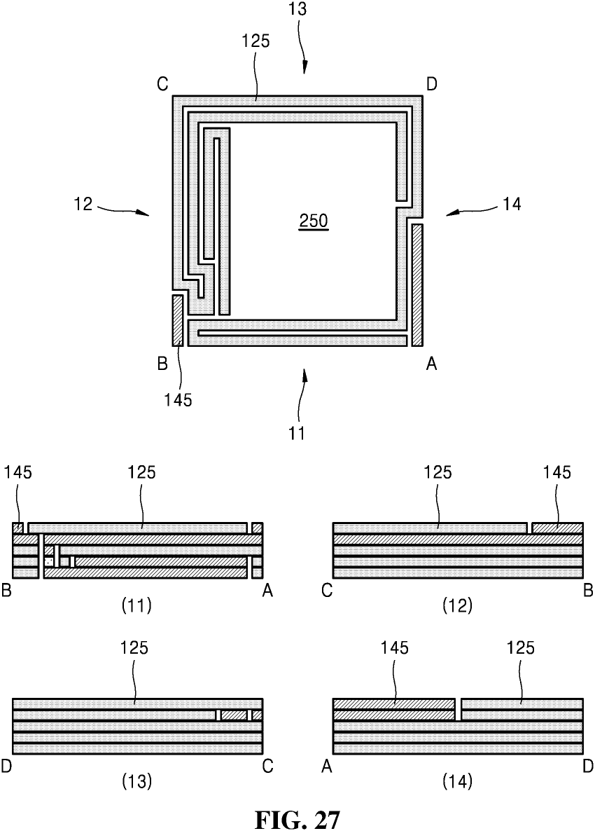

Referring to FIG. 26, a fourth layer 240 is formed. The fourth layer 240 may be formed by forming a fourth insulator region 124 and a fourth conductor region 144.

On the first side surface 11, the fourth conductor region 144 may extend at an outermost side, and be formed as one or more lines. The fourth insulator region 124 may extend at an inner side of the fourth conductor region 144, and be formed as one or more lines.

On the second side surface 12, the fourth conductor region 144 may extend at an outermost side, and be formed as one or more lines. The fourth insulator region 124 may extend at an inner side of the fourth conductor region 144, and be formed as one or more lines.

On the third side surface 13, the fourth insulator region 124 may extend at an outermost side, and be formed as one or more lines.

On the fourth side surface 14, the fourth insulator region 124 may be formed at a partial region of an outermost side, and be formed as one or more lines. The fourth conductor region 144 may be formed at another partial region of the outermost side, and be formed as one or more lines. The fourth insulator region 124 may extend at an inner side of the fourth conductor region 144, and be formed as one or more lines.

The fourth conductor region 144 formed on the first, second, and fourth side surfaces 11, 12, and 14 and the fourth insulator region 124 formed on the third and fourth side surfaces 13 and 14 may form outer walls.

The third conductor region 143 of the third layer 230 and the fourth conductor region 144 of the fourth layer 240, which are in contact with each other, may be bonded and electrically connected to each other. The third insulator region 123 of the third layer 230 and the fourth insulator region 124 of the fourth layer 240, which are in contact with each other, may be bonded to each other through sintering or the like.

Referring to FIG. 27, a fifth layer 250 is formed. The fifth layer 250 may be formed by forming a fifth insulator region 125 and a fifth conductor region 145.

On the first side surface 11, the fifth insulator region 125 may extend at an outermost side, and be formed as one or more lines.

On the second side surface 12, the fifth conductor region 145 may extend at a partial region of an outermost side, and be formed as one or more lines. The fifth insulator region 125 may be formed at another partial region of the outermost side, and be formed as one or more lines. The fifth insulator region 125 may extend at an inner side of the fifth conductor region 145, and be formed as one or more lines.

On the third side surface 13, the fifth insulator region 125 may extend at an outermost side, and be formed as one or more lines.

On the fourth side surface 14, the fifth insulator region 125 may be formed at a partial region of an outermost side, and be formed as one or more lines. The fifth conductor region 145 may be formed at another partial region of the outermost side, and be formed as one or more lines. The fifth insulator region 125 may extend at an inner side of the fifth conductor region 145, and be formed as one or more lines.

The fifth conductor region 145 formed on the second and fourth side surfaces 12 and 14 and the fifth insulator region 125 formed on the first to fourth side surfaces 11 to 14 may form outer walls.

The fourth conductor region 144 of the fourth layer 240 and the fifth conductor region 145 of the fifth layer 250, which are in contact with each other, may be bonded and electrically connected to each other. The fourth insulator region 124 of the fourth layer 240 and the fifth insulator region 125 of the fifth layer 250, which are in contact with each other, may be bonded to each other through sintering or the like.

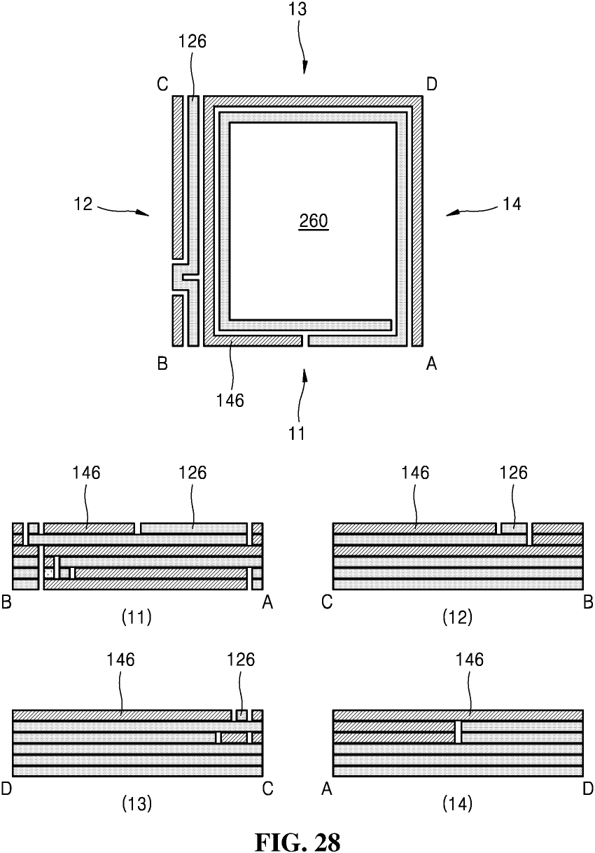

Referring to FIG. 28, a sixth layer 260 is formed. The sixth layer 260 may be formed by forming a sixth insulator region 126 and a sixth conductor region 146.

On the first side surface 11, the sixth conductor region 146 may extend at a partial region of an outermost side, and be formed as one or more lines. The sixth insulator region 126 may be formed at another partial region of the outermost side, and be formed as one or more lines. The sixth insulator region 126 may extend at an inner side of the sixth conductor region 146, and be formed as one or more lines.

On the second side surface 12, the sixth conductor region 146 may extend at a partial region of an outermost side, and be formed as one or more lines. The sixth conductor region 146 may be divided into two separate portions. The sixth insulator region 126 may be disposed at another partial region of the outermost side to divide the sixth conductor region 146. The sixth insulator region 126 may extend at an inner side of the sixth conductor region 146, and be formed as one or more lines. The sixth conductor region 146 may extend at an inner side of the sixth insulator region 126. The sixth insulator region 126 may extend at an inner side of the sixth conductor region 146. That is, the sixth conductor region 146 and the sixth insulator region 126 may be alternately disposed.

On the third side surface 13, the sixth conductor region 146 may extend at an outermost side, and be formed as one or more lines. The sixth insulator region 126 may extend at an inner side of the sixth conductor region 146, and be formed as one or more lines.

On the fourth side surface 14, the sixth conductor region 146 may extend at an outermost side, and be formed as one or more lines. The sixth insulator region 126 may extend at an inner side of the sixth conductor region 146, and be formed as one or more lines.

The sixth conductor region 146 formed on the first to fourth side surfaces 11 to 14 and the sixth insulator region 126 formed on the first side surface 11 may form outer walls.

The fifth conductor region 145 of the fifth layer 250 and the sixth conductor region 146 of the sixth layer 260, which are in contact with each other, may be bonded and electrically connected to each other. The fifth insulator region 125 of the fifth layer 250 and the sixth insulator region 126 of the sixth layer 260, which are in contact with each other, may be bonded to each other through sintering or the like.

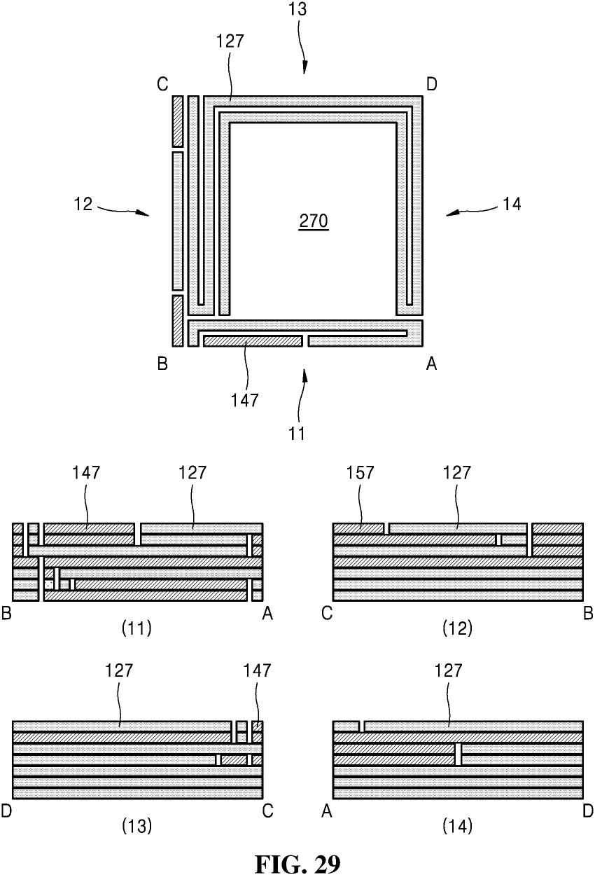

Referring to FIG. 29, a seventh layer 270 is formed. The seventh layer 270 may be formed by forming a seventh insulator region 127 and a seventh conductor region 147.

On the first side surface 11, the seventh conductor region 147 may extend at a partial region of an outermost side, and be formed as one or more lines. The seventh insulator region 127 may be formed at another partial region of the outermost side, and be formed as one or more lines. The seventh insulator region 127 may extend at an inner side of the seventh conductor region 147, and be formed as one or more lines.

On the second side surface 12, the seventh conductor region 147 may extend at a partial region of an outermost side, and be formed as one or more lines. The seventh conductor region 147 may be divided into two separate portions. The seventh insulator region 127 may be disposed at another partial region of the outermost side to divide the seventh conductor region 147. The seventh insulator region 127 may extend at an inner side of the seventh conductor region 147, and be formed as one or more lines.

On the third side surface 13, the seventh insulator region 127 may extend at an outermost side, and be formed as one or more lines.

On the fourth side surface 14, the seventh insulator region 127 may extend at an outermost side, and be formed as one or more lines.

The seventh conductor region 147 formed on the first side surface 11 and the seventh insulator region 127 formed on the first to fourth side surfaces 11 to 14 may form outer walls.

The sixth conductor region 146 of the sixth layer 260 and the seventh conductor region 147 of the seventh layer 270, which are in contact with each other, may be bonded and electrically connected to each other. The sixth insulator region 126 of the sixth layer 260 and the seventh insulator region 127 of the seventh layer 270, which are in contact with each other, may be bonded to each other through sintering or the like.

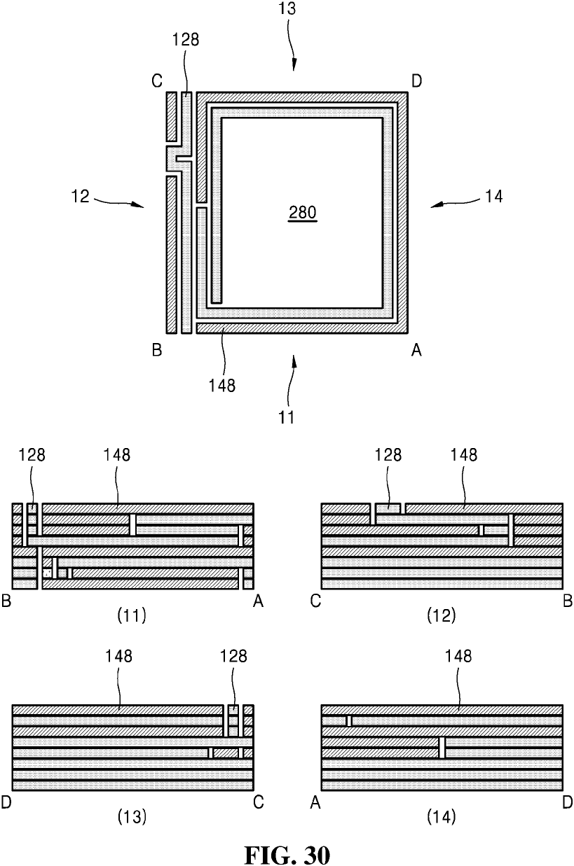

Referring to FIG. 30, an eighth layer 280 is formed. The eighth layer 280 may be formed by forming an eighth insulator region 128 and an eighth conductor region 148.

On the first side surface 11, the eighth conductor region 148 may extend at an outermost side, and be formed as one or more lines. The eighth insulator region 128 may extend at an inner side of the eighth conductor region 148, and be formed as one or more lines.

On the second side surface 12, the eighth conductor region 148 may extend at a partial region of an outermost side, and be formed as one or more lines. The eighth conductor region 148 may be divided into two separate portions. The eighth insulator region 128 may be disposed at another partial region of the outermost side to divide the eighth conductor region 148. The eighth insulator region 128 may extend at an inner side of the eighth conductor region 148, and be formed as one or more lines. The eighth conductor region 148 may extend at a partial region of an inner side of the eighth insulator region 128. The eighth insulator region 128 may extend at an inner side of the eighth conductor region 148. That is, the eighth conductor region 148 and the eighth insulator region 128 may be alternately disposed.

On the third side surface 13, the eighth conductor region 148 may extend at an outermost side, and be formed as one or more lines. The eighth insulator region 128 may extend at an inner side of the eighth conductor region 148, and be formed as one or more lines.

On the fourth side surface 14, the eighth conductor region 148 may extend at an outermost side, and be formed as one or more lines. The eighth insulator region 128 may extend at an inner side of the eighth conductor region 148, and be formed as one or more lines.

The eighth conductor region 148 formed on the first to fourth side surfaces 11 to 14 and the eighth insulator region 128 formed on the second side surface 12 may form outer walls.

The seventh conductor region 147 of the seventh layer 270 and the eighth conductor region 148 of the eighth layer 280, which are in contact with each other, may be bonded and electrically connected to each other. The seventh insulator region 127 of the seventh layer 270 and the eighth insulator region 128 of the eighth layer 280, which are in contact with each other, may be bonded to each other through sintering or the like.

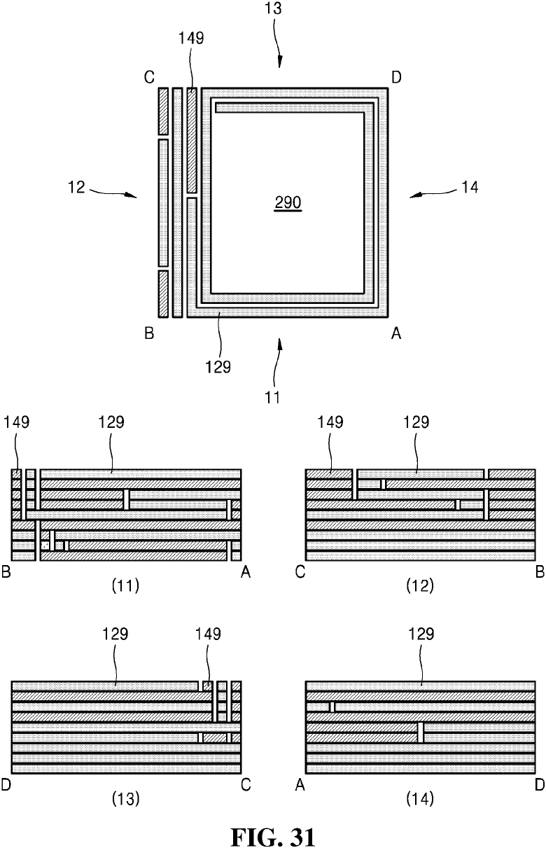

Referring to FIG. 31, a ninth layer 290 is formed. The ninth layer 290 may be formed by forming a ninth insulator region 129 and a ninth conductor region 149.

On the first side surface 11, the ninth insulator region 129 may extend at an outermost side, and be formed as one or more lines.

On the second side surface 12, the ninth conductor region 149 may extend at a partial region of an outermost side, and be formed as one or more lines. The ninth conductor region 149 may be divided into two separate portions. The ninth insulator region 129 may be disposed at another partial region of the outermost side to divide the ninth conductor region 149. The ninth insulator region 129 may extend at an inner side of the ninth conductor region 149, and be formed as one or more lines. The ninth conductor region 149 may extend at a partial region of an inner side of the ninth insulator region 129. The ninth insulator region 129 may extend at an inner side of the ninth conductor region 149. That is, the ninth conductor region 149 and the ninth insulator region 129 may be alternately disposed.

On the third side surface 13, the ninth insulator region 129 may extend at an outermost side, and be formed as one or more lines.

On the fourth side surface 14, the ninth insulator region 129 may extend at an outermost side, and be formed as one or more lines.

The ninth conductor region 149 formed on the second side surface 12 and the ninth insulator region 129 formed on the first to fourth side surfaces 11 to 14 may form outer walls.

The eighth conductor region 148 of the eighth layer 280 and the ninth conductor region 149 of the ninth layer 290, which are in contact with each other, may be bonded and electrically connected to each other. The eighth insulator region 128 of the eighth layer 280 and the ninth insulator region 129 of the ninth layer 290, which are in contact with each other, may be bonded to each other through sintering or the like.

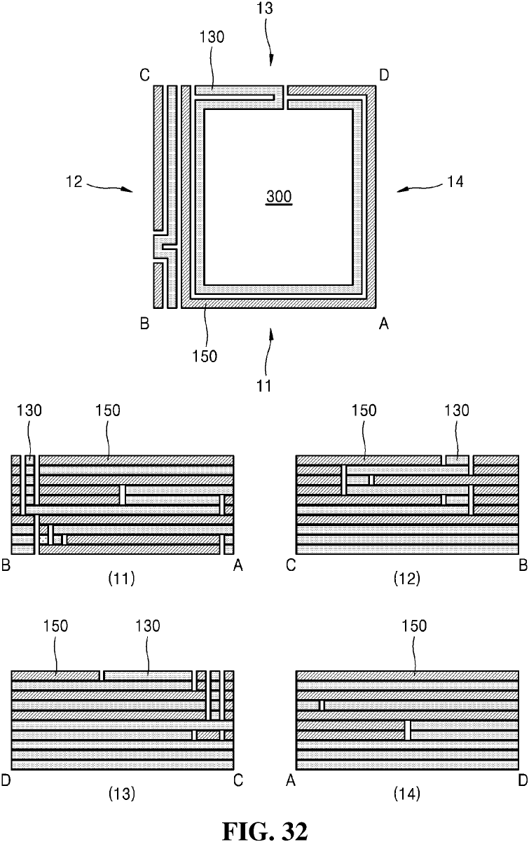

Referring to FIG. 32, a tenth layer 300 is formed. The tenth layer 300 may be formed by forming a tenth insulator region 130 and a tenth conductor region 150.

On the first side surface 11, the tenth conductor region 150 may extend at an outermost side, and be formed as one or more lines. The tenth insulator region 130 may extend at an inner side of the tenth conductor region 150, and be formed as one or more lines.

On the second side surface 12, the tenth conductor region 150 may extend at a partial region of an outermost side, and be formed as one or more lines. The tenth conductor region 150 may be divided into two separate portions. The tenth insulator region 130 may be disposed at another partial region of the outermost side to divide the tenth conductor region 150. The tenth insulator region 130 may extend at an inner side of the tenth conductor region 150, and be formed as one or more lines. The tenth conductor region 150 may extend at an inner side of the tenth insulator region 130. The tenth insulator region 130 may extend at an inner side of the tenth conductor region 150. That is, the tenth conductor region 150 and the tenth insulator region 130 may be alternately disposed.

On the third side surface 13, the tenth insulator region 130 may be formed at a partial region of an outermost side, and be formed as one or more lines. The tenth conductor region 150 may be formed at another partial region of the outermost side, and be formed as one or more lines. The tenth insulator region 130 may extend at an inner side of the tenth conductor region 150, and be formed as one or more lines.

On the fourth side surface 14, the tenth conductor region 150 may extend at an outermost side, and be formed as one or more lines. The tenth insulator region 130 may extend at an inner side of the tenth conductor region 150, and be formed as one or more lines.

The tenth conductor region 150 formed on the first to fourth side surfaces 11 to 14 and the tenth insulator region 130 formed on the second and third side surfaces 12 and 13 may form outer walls.

The ninth conductor region 149 of the ninth layer 290 and the tenth conductor region 150 of the tenth layer 300, which are in contact with each other, may be bonded and electrically connected to each other. The ninth insulator region 129 of the ninth layer 290 and the tenth insulator region 130 of the tenth layer 300, which are in contact with each other, may be bonded to each other through sintering or the like.

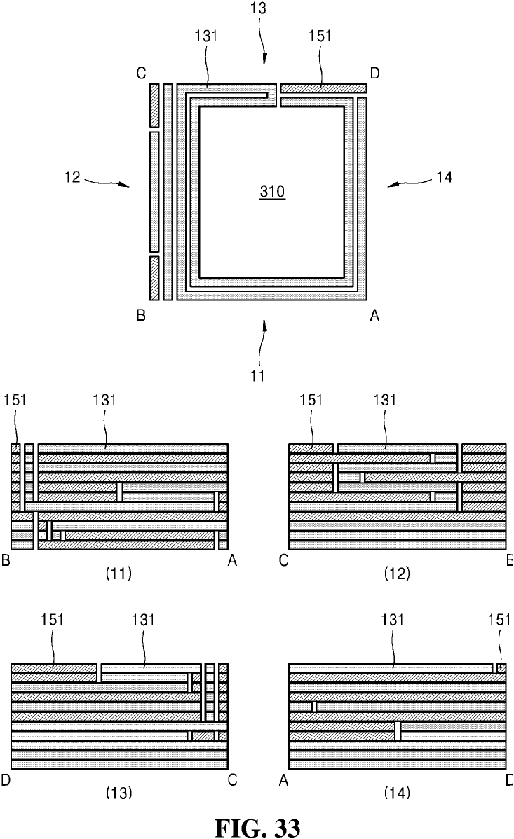

Referring to FIG. 33, an eleventh layer 310 is formed. The eleventh layer 310 may be formed by forming an eleventh insulator region 131 and an eleventh conductor region 151.

On the first side surface 11, the eleventh insulator region 131 may extend at an outermost side, and be formed as one or more lines.

On the second side surface 12, the eleventh conductor region 151 may extend at a partial region of an outermost side, and be formed as one or more lines. The eleventh conductor region 151 may be divided into two separate portions. The eleventh insulator region 131 may be disposed at another partial region of the outermost side to divide the eleventh conductor region 151. The eleventh insulator region 131 may extend at an inner side of the eleventh conductor region 151, and be formed as one or more lines.

On the third side surface 13, the eleventh insulator region 131 may be formed at a partial region of an outermost side, and be formed as one or more lines. The eleventh conductor region 151 may be formed at another partial region of the outermost side, and be formed as one or more lines. The eleventh insulator region 131 may extend at an inner side of the eleventh conductor region 151, and be formed as one or more lines.

On the fourth side surface 14, the eleventh insulator region 131 may extend at an outermost side, and be formed as one or more lines.

The eleventh conductor region 151 formed on the second and third side surfaces 12 and 13 and the eleventh insulator region 131 formed on the first to fourth side surfaces 11 to 14 may form outer walls.

The tenth conductor region 150 of the tenth layer 300 and the eleventh conductor region 151 of the eleventh layer 310, which are in contact with each other, may be bonded and electrically connected to each other. The tenth insulator region 130 of the tenth layer 300 and the eleventh insulator region 131 of the eleventh layer 310, which are in contact with each other, may be bonded to each other through sintering or the like.

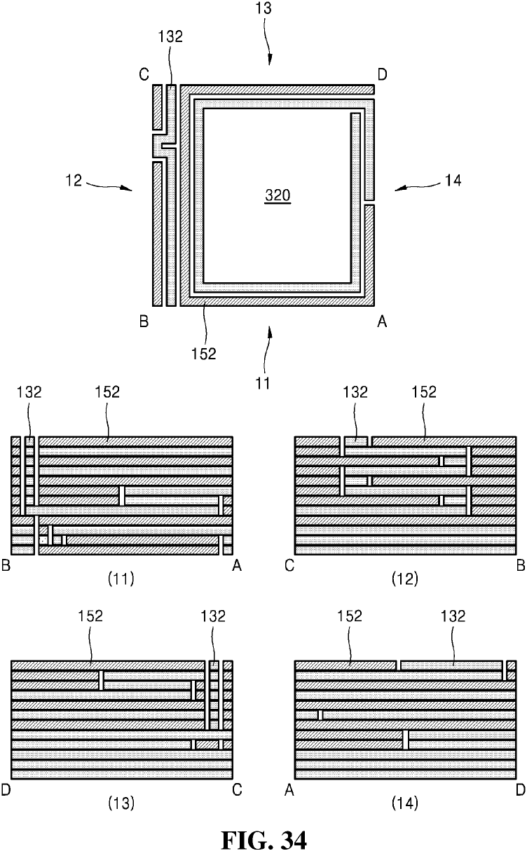

Referring to FIG. 34, a twelfth layer 320 is formed. The twelfth layer 320 may be formed by forming a twelfth insulator region 132 and a twelfth conductor region 152.

On the first side surface 11, the twelfth conductor region 152 may extend at an outermost side, and be formed as one or more lines. The twelfth insulator region 132 may extend at an inner side of the twelfth conductor region 152, and be formed as one or more lines.

On the second side surface 12, the twelfth conductor region 152 may extend at a partial region of an outermost side, and be formed as one or more lines. The twelfth conductor region 152 may be divided into two separate portions. The twelfth insulator region 132 may be disposed at another partial region of the outermost side to divide the twelfth conductor region 152. The twelfth insulator region 132 may extend at an inner side of the twelfth conductor region 152, and be formed as one or more lines. The twelfth conductor region 152 may extend at an inner side of the twelfth insulator region 132. The twelfth insulator region 132 may extend at an inner side of the twelfth conductor region 152. That is, the twelfth conductor region 152 and the twelfth insulator region 132 may be alternately disposed.

On the third side surface 13, the twelfth conductor region 152 may extend at an outermost side, and be formed as one or more lines. The twelfth insulator region 132 may extend at an inner side of the twelfth conductor region 152, and be formed as one or more lines.

On the fourth side surface 14, the twelfth insulator region 132 may be formed at a partial region of an outermost side, and be formed as one or more lines. The twelfth conductor region 152 may be formed at another partial region of the outermost side, and be formed as one or more lines. The twelfth insulator region 132 may extend at an inner side of the twelfth conductor region 152, and be formed as one or more lines.

The twelfth conductor region 152 formed on the first to fourth side surfaces 11 to 14 and the twelfth insulator region 132 formed on the second and fourth side surfaces 12 and 14 may form outer walls.

The eleventh conductor region 151 of the eleventh layer 310 and the twelfth conductor region 152 of the twelfth layer 320, which are in contact with each other, may be bonded and electrically connected to each other. The eleventh insulator region 131 of the eleventh layer 310 and the twelfth insulator region 132 of the twelfth layer 320, which are in contact with each other, may be bonded to each other through sintering or the like.

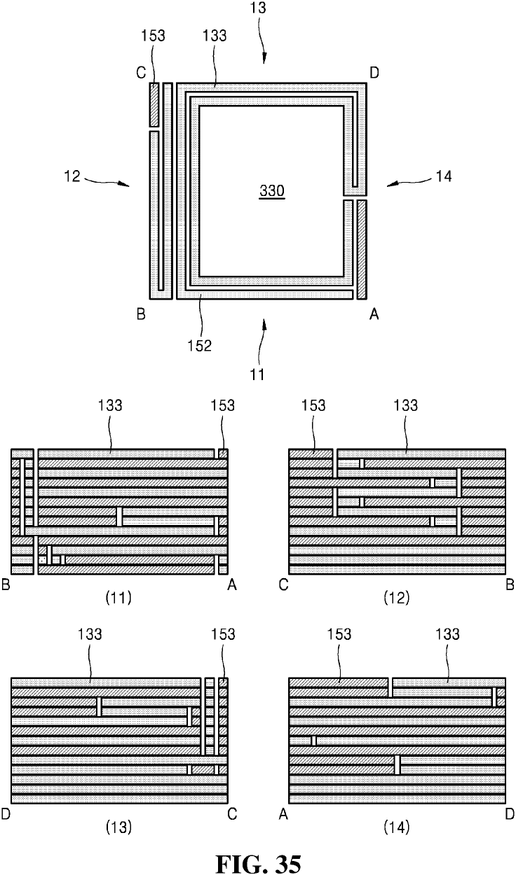

Referring to FIG. 35, a thirteenth layer 330 is formed. The thirteenth layer 330 may be formed by forming a thirteenth insulator region 133 and a thirteenth conductor region 153.

On the first side surface 11, the thirteenth insulator region 133 may extend at an outermost side, and be formed as one or more lines.

On the second side surface 12, the thirteenth conductor region 153 may extend at a partial region of an outermost side, and be formed as one or more lines. The thirteenth insulator region 133 may be formed at another partial region of the outermost side, and be formed as one or more lines. The thirteenth insulator region 133 may extend at an inner side of the thirteenth conductor region 153, and be formed as one or more lines.

On the third side surface 13, the thirteenth insulator region 133 may extend at an outermost side, and be formed as one or more lines.

On the fourth side surface 14, the thirteenth insulator region 133 may be formed at a partial region of an outermost side, and be formed as one or more lines. The thirteenth conductor region 153 may be formed at another partial region of the outermost side, and be formed as one or more lines. The thirteenth insulator region 133 may extend at an inner side of the thirteenth conductor region 153, and be formed as one or more lines.

The thirteenth conductor region 153 formed on the second and fourth side surfaces 12 and 14 and the thirteenth insulator region 133 formed on the first to fourth side surfaces 11 to 14 may form outer walls.

The twelfth conductor region 152 of the twelfth layer 320 and the thirteenth conductor region 153 of the thirteenth layer 330, which are in contact with each other, may be bonded and electrically connected to each other. The twelfth insulator region 132 of the twelfth layer 320 and the thirteenth insulator region 133 of the thirteenth layer 330, which are in contact with each other, may be bonded to each other through sintering or the like.

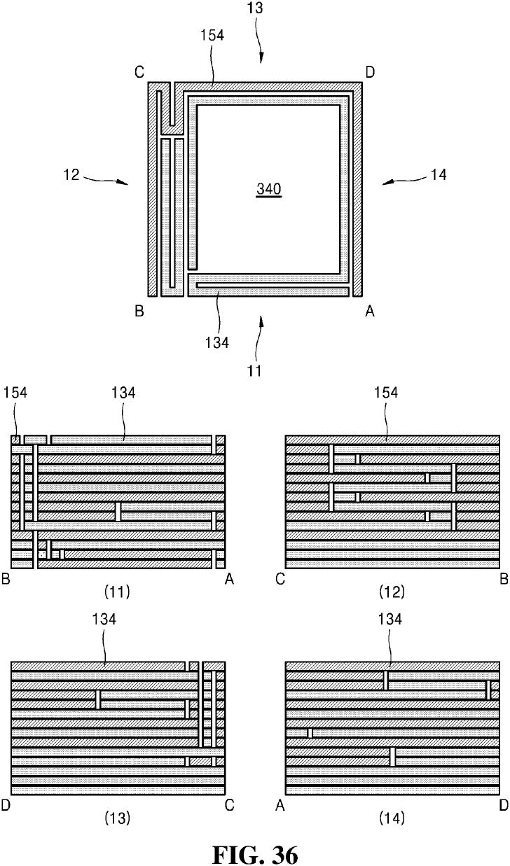

Referring to FIG. 36, a fourteenth layer 340 is formed. The fourteenth layer 340 may be formed by forming a fourteenth insulator region 134 and a fourteenth conductor region 154.

On the first side surface 11, the fourteenth insulator region 134 may extend at an outermost side, and be formed as one or more lines.

On the second side surface 12, the fourteenth conductor region 154 may extend at an outermost side, and be formed as one or more lines. The fourteenth insulator region 134 may extend at an inner side of the fourteenth conductor region 154, and be formed as one or more lines.

On the third side surface 13, the fourteenth conductor region 154 may extend at an outermost side, and be formed as one or more lines. The fourteenth insulator region 134 may extend at an inner side of the fourteenth conductor region 154, and be formed as one or more lines.

On the fourth side surface 14, the fourteenth conductor region 154 may extend at an outermost side, and be formed as one or more lines. The fourteenth insulator region 134 may extend at an inner side of the fourteenth conductor region 154, and be formed as one or more lines.

The fourteenth conductor region 154 formed on the second to fourth side surfaces 12 to 14 and the fourteenth insulator region 134 formed on the first side surface 11 may form outer walls.

The thirteenth conductor region 153 of the thirteenth layer 330 and the fourteenth conductor region 154 of the fourteenth layer 340, which are in contact with each other, may be bonded and electrically connected to each other. The thirteenth insulator region 133 of the thirteenth layer 330 and the fourteenth insulator region 134 of the fourteenth layer 340, which are in contact with each other, may be bonded to each other through sintering or the like.

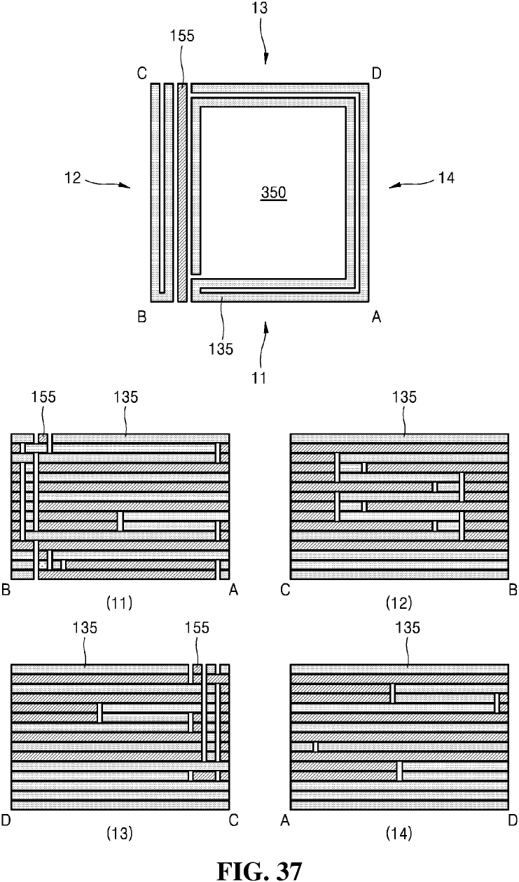

Referring to FIG. 37, a fifteenth layer 350 is formed. The fifteenth layer 350 may be formed by forming a fifteenth insulator region 135 and a fifteenth conductor region 155.

On the first side surface 11, the fifteenth insulator region 135 may extend at an outermost side, and be formed as one or more lines.

On the second side surface 12, the fifteenth insulator region 135 may extend at an outermost side, and be formed as one or more lines. The fifteenth conductor region 155 may extend at an inner side of the fifteenth insulator region 135, and be formed as one or more lines.

On the third side surface 13, the fifteenth insulator region 135 may extend at an outermost side, and be formed as one or more lines.

On the fourth side surface 14, the fifteenth insulator region 135 may extend at an outermost side, and be formed as one or more lines.

The fifteenth insulator region 135 formed on the first to fourth side surfaces 11 to 14 may form outer walls.

The fourteenth conductor region 154 of the fourteenth layer 340 and the fifteenth conductor region 155 of the fifteenth layer 350, which are in contact with each other, may be bonded and electrically connected to each other. The fourteenth insulator region 134 of the fourteenth layer 340 and the fifteenth insulator region 135 of the fifteenth layer 350, which are in contact with each other, may be bonded to each other through sintering or the like.

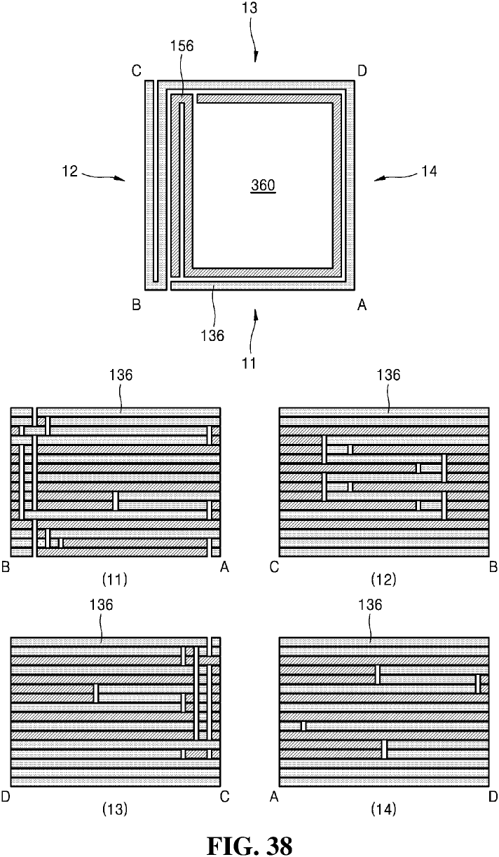

Referring to FIG. 38, a sixteenth layer 360 is formed. The sixteenth layer 360 may be formed by forming a sixteenth insulator region 136 and a sixteenth conductor region 156.

On the first side surface 11, the sixteenth insulator region 136 may extend at an outermost side, and be formed as one or more lines. The sixteenth conductor region 156 may extend at an inner side of the sixteenth insulator region 136, and be formed as one or more lines.

On the second side surface 12, the sixteenth insulator region 136 may extend at an outermost side, and be formed as one or more lines. The sixteenth conductor region 156 may extend at an inner side of the sixteenth insulator region 136, and be formed as one or more lines.

On the third side surface 13, the sixteenth insulator region 136 may extend at an outermost side, and be formed as one or more lines. The sixteenth conductor region 156 may extend at an inner side of the sixteenth insulator region 136, and be formed as one or more lines.

On the fourth side surface 14, the sixteenth insulator region 136 may extend at an outermost side, and be formed as one or more lines. The sixteenth conductor region 156 may extend at an inner side of the sixteenth insulator region 136, and be formed as one or more lines.

The sixteenth insulator region 136 formed on the first to fourth side surfaces 11 to 14 may form outer walls.

The fifteenth conductor region 155 of the fifteenth layer 350 and the sixteenth conductor region 156 of the sixteenth layer 360, which are in contact with each other, may be bonded and electrically connected to each other. The fifteenth insulator region 135 of the fifteenth layer 350 and the sixteenth insulator region 136 of the sixteenth layer 360, which are in contact with each other, may be bonded to each other through sintering or the like.

The first conductor region 141 formed on the first layer 210 and the second conductor region 142 formed on the second layer 220 may configure a lower electrode.

A diode from among the electronic devices of the biodegradable electroceutical 100 may be configured as described below.

The first conductor region 141 formed on the first layer 210, the semiconductor region 152 formed on the second layer 220, and the third conductor region 143 formed on the third layer 230 may be vertically aligned to configure a diode.

A capacitor from among the electronic devices of the biodegradable electroceutical 100 may be configured as described below.

The fourth conductor region 144 formed on the fourth layer 240, the fifth insulator region 125 formed on the fifth layer 250, and the sixth conductor region 146 formed on the sixth layer 260 may be vertically aligned on the second side surface 12 to configure a capacitor.

The sixth conductor region 146 formed on the sixth layer 260, the seventh insulator region 127 formed on the seventh layer 270, and the eighth conductor region 148 formed on the eighth layer 280 may be vertically aligned on the second side surface 12 to configure a capacitor.

The eighth conductor region 148 formed on the eighth layer 280, the ninth insulator region 129 formed on the ninth layer 290, and the tenth conductor region 150 formed on the tenth layer 300 may be vertically aligned on the second side surface 12 to configure a capacitor.

The tenth conductor region 150 formed on the tenth layer 300, the eleventh insulator region 131 formed on the eleventh layer 310, and the twelfth conductor region 152 formed on the twelfth layer 320 may be vertically aligned on the second side surface 12 to configure a capacitor.

The twelfth conductor region 152 formed on the twelfth layer 320, the thirteenth insulator region 133 formed on the thirteenth layer 330, and the fourteenth conductor region 154 formed on the fourteenth layer 340 may be vertically aligned on the second side surface 12 to configure a capacitor.

The capacitors may be alternately engaged with each other.

The fourth, eighth, and twelfth conductor regions 144, 148, and 152 may be electrically connected to each other. For example, the fourth conductor region 144 may be electrically connected to the eighth conductor region 148 through the fifth conductor region 145, another portion of the sixth conductor region 146, and the seventh conductor region 147. The eighth conductor region 148 may be electrically connected to the twelfth conductor region 152 through the ninth conductor region 149, another portion of the tenth conductor region 150, and the eleventh conductor region 151.

The sixth, tenth, and fourteenth conductor regions 146, 150, and 154 may be electrically connected to each other. For example, the sixth conductor region 146 may be electrically connected to the tenth conductor region 150 through the seventh conductor region 147, another portion of the eighth conductor region 148, and the ninth conductor region 149. The tenth conductor region 150 may be electrically connected to the fourteenth conductor region 154 through the eleventh conductor region 151, another portion of the twelfth conductor region 152, and the thirteenth conductor region 153.

An inductor from among the electronic devices of the biodegradable electroceutical 100 may be configured as described below.

The fourth to fourteenth conductor regions 144 to 154 formed on the fourth to fourteenth layers 240 to 340 may be vertically aligned and wound around the hollow part in the same direction to configure a vertically extending inductor.

The fifteenth conductor region 155 formed on the fifteenth layer 350 and the sixteenth conductor region 156 formed on the sixteenth layer 360 may configure an upper electrode.

The lower and upper electrodes may function as the first and second wires 108 and 109 of FIG. 20, and be in contact with the target nerve cell NC.

Each of the first to sixteenth layers 210 to 360 may include one or more layers.

Then, the biodegradable electroceutical 100 may be formed by bonding the plurality of layers to each other by using at least one of heat treatment, light irradiation, chemical treatment, and electrochemical treatment. For example, the insulator and semiconductor regions may be bonded using sintering, and the conductor regions may be bonded using melting or alloying.

According to the electrochemical treatment, the conductor regions may be made of a conductive material filler and a surface oxide layer, the surface oxide layer may be reduced and decomposed by oxidation-reduction reaction with an acidic catalyst, and particles of the conductive material filler may be bonded to each other to form a conductive network. The above bonding method may also be applied to the semiconductor region.

At least one of the above-described conductor, insulator, and semiconductor regions may include a biodegradable metal material. The biodegradable material refers to a material that may be absorbed into the human body and is harmless after absorption. The conductor regions may include, for example, Mg, Fe, Zn, Mo, W, Ca, K, Na, Si, a-IGZO, Ge, or an alloy thereof. The insulator regions may include, for example, oxide of Mg, Fe, Zn, Mo, W, Ca, K, Na, Si, a-IGZO, Ge, or an alloy thereof. The semiconductor region may include a material obtained by doping a conductive material on the material of the insulator regions. For example, the semiconductor region may include AZO.

A biodegradable electroceutical according to an embodiment of the present invention may be formed using the 3D printing method described above in relation to FIG. 16.

According to the present invention, a biodegradable electronic device or a biodegradable electroceutical in the medical industry may have embedded therein an electrical circuit formed by 3D-printing a biodegradable conductive, semiconductive, dielectric, or insulating paste. As such, a passively controllable structure may be actively controlled by adding electronic functions such as wireless communication or electrostimulation to a simple structure. For example, an implantable biodegradable electroceutical including a circuit and electrodes capable of providing electrostimulation in a wireless manner may promote healing and regeneration of cells in the human body, or be provided with a pressure sensor or a strain sensor to pioneer a new field of monitoring an applied pressure or stress while being implanted in the human body. The biodegradable electroceutical may also be applied to biotechnology such as food and agriculture. Specifically, as an implantable medical device, the biodegradable electroceutical may promote regeneration of peripheral nerves by using a customized biodegradable electrostimulator, or promote differentiation through electrostimulation by inserting stem cells into an electrode-embedded biodegradable scaffold.

According to the present invention, a method of forming the biodegradable electroceutical, and more specifically, a 3D stacking method using a 3D printer or the like, may be applied to smart food packaging and used as a sensor for measuring humidity, temperature, or impact inside fruit packaging. In addition, the biodegradable electroceutical may be applied to smart farms and used as a sensor for measuring pH, moisture, or temperature in the soil, or temperature, humidity, or fine dusts in the air.

While the present invention has been particularly shown and described with reference to embodiments thereof, it will be understood by one of ordinary skill in the art that various changes in form and details may be made therein without departing from the scope of the present invention as defined by the following claims.

### INDUSTRIAL APPLICABILITY

The biodegradable electroceutical according to the present invention may promote healing and regeneration of cells in the human body, or be provided with a pressure sensor or a strain sensor to pioneer a new field of monitoring an applied pressure or stress while being implanted in the human body. The biodegradable electroceutical may also be applied to biotechnology such as food and agriculture.

1-40. (canceled)

41. A paste for producing biodegradable electroceuticals, the paste comprising:
a functional inorganic powder providing conductive, semiconductive, dielectric, or insulating properties;
a humectant;
a matrix polymer; and
an organic solvent.

42. The paste of claim 41, wherein the functional inorganic powder provides a conductive function, and comprises one or more selected from the group consisting of magnesium (Mg), iron (Fe), zinc (Zn), molybdenum (Mo), tungsten (W), calcium (Ca), potassium (K), sodium (Na), silicon (Si), amorphous indium gallium zinc oxide (a-IGZO), germanium (Ge), and alloys thereof.

43. The paste of claim 41, wherein the functional inorganic powder provides a conductive function,
wherein a volume fraction of the functional inorganic powder with respect to a total volume of the paste ranges from 0.35 to 0.41, and
wherein the paste comprises:
1 g to 15 g of the functional inorganic powder;
0.1 ml to 1 ml of the humectant; and
0.05 g to 1 g of the matrix polymer, per 1 ml of the organic solvent.

44. The paste of claim 41, wherein the functional inorganic powder provides a semiconductive function, and comprises one or more selected from the group consisting of Si, Ge, zinc oxide (ZnO), and aluminum-doped zinc oxide (AZO).

45. The paste of claim 41, wherein the functional inorganic powder provides a semiconductive function,
wherein a volume fraction of the functional inorganic powder with respect to a total volume of the paste ranges from 0.17 to 0.23, and
wherein the paste comprises:
1 g to 5 g of the functional inorganic powder;
0.1 ml to 1 ml of the humectant; and
0.05 g to 1 g of the matrix polymer, per 1 ml of the organic solvent.

46. The paste of claim 41, wherein the functional inorganic powder provides a dielectric or insulating function, and comprises one or more selected from the group consisting of Mg oxide, Fe oxide, Zn oxide, Mo oxide, W oxide, Ca oxide, K oxide, Na oxide, Si oxide, Ge oxide, Mg nitride, Fe nitride, Zn nitride, Mo nitride, W nitride, Ca nitride, K nitride, Na nitride, Si nitride, Ge nitride, and a-IGZO.

47. The paste of claim 41, wherein the functional inorganic powder provides a dielectric or insulating function,
wherein a volume fraction of the functional inorganic powder with respect to a total volume of the paste ranges from 0.05 to 0.08, and
wherein the paste comprises:
0.1 g to 2 g of the functional inorganic powder;
0.1 ml to 1 ml of the humectant; and
0.05 g to 1 g of the matrix polymer, per 1 ml of the organic solvent.

48. The paste of claim 41, wherein the humectant comprises one or more selected from the group consisting of tetraglycol (TG), ethylene glycol, and N-methyl-2-pyrrolidone (NMP),
wherein the matrix polymer comprises one or more selected from the group consisting of polycaprolactone (PCL), silk fibroin, sodium carboxymethyl cellulose (Na-CMC), polyvinyl alcohol (PVA), polyvinylpyrrolidone (PVP), poly lactic-co-glycolic acid (PLGA), polylactic acid (PLA), polyglycerol sebacate (PGS), and polybutylene adipate terephthalate (PBAT), and
wherein the organic solvent comprises one or more selected from the group consisting of tetrahydrofuran (THF), dichloromethane (DCM), chloroform, dimethylformamide (DMF), acetone, and ethyl acetate.

49. The paste of claim 41, wherein the paste has a viscosity ranging from 50 Pa·s to 1000 Pa·s at a shear rate ranging from 1 s⁻¹ to 100 /s⁻¹, and has a yield shear stress ranging from 10² Pa to 10³ Pa at a shear strain ranging from 0.01% to 10%.

50. The paste of claim 41, wherein at least one of the functional inorganic powder, the humectant, the matrix polymer, and the organic solvent is made of a biodegradable material that is decomposed in a human body.

51. A method of producing a biodegradable electronic device by using a paste for producing biodegradable electroceuticals, the method comprising:
providing the paste comprising at least one of conductive, semiconductive, dielectric, and insulating properties;
forming an electronic device structure by using the paste; and
forming a biodegradable electronic device by providing conductive properties by sintering the electronic device structure,
wherein the providing of the paste comprises forming the paste, by mixing a functional inorganic powder providing conductive, semiconductive, dielectric, or insulating properties, a humectant, a matrix polymer, and an organic solvent at the above-mentioned fractions.

52. The method of claim 51, wherein the forming of the electronic device structure is performed by three-dimensionally (3D) printing the paste, and
wherein the forming of the biodegradable electronic device is performed through sintering based on heat treatment, light irradiation, chemical treatment, or electrochemical treatment.

53. A biodegradable electroceutical comprising:
a plurality of material layers comprising at least one of an insulator region, a conductor region, and a semiconductor region, and stacked on one another;
one or more electronic devices configured by a combination of the plurality of material layers; and
a hollow part provided in middle of the plurality of material layers to insert a nerve cell thereinto,
wherein the electronic devices are formed using the method according to claim 51, and comprise at least one of a diode, a capacitor, an inductor, a resistor, a transistor, an electrode, a rectifier, a switch, a memory, a condenser, and a vibrator.

54. The biodegradable electroceutical of claim 53, wherein the electronic devices are configured over the plurality of material layers in a vertical direction.

55. The biodegradable electroceutical of claim 53, wherein the material layers comprise:
a first layer comprising a first conductor region;
a second layer comprising a semiconductor region or an insulator region; and
a third layer comprising a second conductor region,
wherein the first to third layers are sequentially stacked on one another, and
wherein the first conductor region, the semiconductor region or an insulator region, and the second conductor region are vertically aligned to configure a diode or a capacitor as the electronic device.

56. The biodegradable electroceutical of claim 53, wherein the material layers comprise:
a first layer comprising a first conductor region;
a second layer comprising a first insulator region;
a third layer comprising a second conductor region;
a fourth layer comprising a second insulator region;
a fifth layer comprising a third conductor region;
a sixth layer comprising a third insulator region; and
a seventh layer comprising a fourth conductor region,
wherein the first to seventh layers are sequentially stacked on one another,
wherein the first conductor region is electrically connected to the third conductor region,
wherein the second conductor region is electrically connected to the fourth conductor region,
wherein the first conductor region, the first insulator region, and the second conductor region are vertically aligned to configure a first capacitor,
wherein the second conductor region, the second insulator region, and the third conductor region are vertically aligned to configure a second capacitor,
wherein the third conductor region, the third insulator region, and the fourth conductor region are vertically aligned to configure a third capacitor, and
optionally, wherein the first and second capacitors or the second and third capacitors are alternately engaged with each other.

57. The biodegradable electroceutical of claim 53, wherein the material layers comprise:
a first layer comprising a first conductor region, and a first insulator region disposed not to connect both ends of the first conductor region;
a second layer comprising a second conductor region in contact with an end of the first conductor region, and a second insulator region disposed to cover and insulate a remaining portion of the first conductor region; and
a third layer comprising a third conductor region in contact with an end of the second conductor region, a third insulator region disposed not to connect both ends of the first conductor region,
wherein the first to third layers are sequentially stacked on one another,
wherein the first to third conductor regions are vertically disposed to configure an inductor as the electronic device, and
optionally, wherein the first to third conductor regions are wound around the hollow part in a same direction.

58. The biodegradable electroceutical of claim 53, wherein the electronic devices comprise a diode, a capacitor, and an inductor,
wherein the capacitor and the inductor are connected in parallel to each other and connected in series to the diode, and
optionally, wherein the capacitor and the inductor are insulated from each other by the insulator region formed in the plurality of material layers.

59. The biodegradable electroceutical of claim 58, further comprising an upper electrode electrically connecting an uppermost side of the capacitor to an uppermost side of the inductor and a lower electrode electrically connecting lowermost sides of the diode.

60. The method of claim 53, wherein the plurality of material layers are stacked on one another by forming, on a previously formed material layer, another material layer by discharging at least one material from among a conductor, an insulator, and a semiconductor directly on the previously formed material layer by using a 3D printer.
